(19) United States
(12) Patent Application Publication
(10) Pub. No.: US 2025/0214973 A1
(43) Pub. Date: Jul. 3, 2025

(54) 1,4,5-TRISUBSTITUTED-1,2,3-TRIAZOLES AND USES THEREOF

(71) Applicant: UNIVERSITY OF PUERTO RICO, San Juan, PR (US)

(72) Inventors: Eliud HERNANDEZ, San Juan, PR (US); Cornelis P. VLAAR, San Juan, PR (US); Suranganie DHARMAWARDHANE, San Juan, PR (US)

(21) Appl. No.: 18/867,293

(22) PCT Filed: May 19, 2023
(86) PCT No.: PCT/US2023/022924

§ 371 Date: Nov. 19, 2024

Related U.S. Application Data
(60) Provisional application No. 63/344,055, filed on May 20, 2022.
(60) Provisional application No. 63/464,018, filed on May 4, 2023.

(51) Int. Cl. C07D 403/04; A61K 31/4192; A61K 31/4439; A61K 31/5377; A61P 35/00; C07D 401/14; C07D 405/14; C07D 409/14

(52) U.S. Cl. CPC C07D 403/04; A61K 31/4192; A61K 31/4439; A61K 31/5377; A61P 35/00; C07D 401/14; C07D 405/14; C07D 409/14

(57) ABSTRACT

The present disclosure relates to cancer metastasis inhibitors, pharmaceutical compositions containing the compounds, and methods of using such compounds to treat disease, such as cancer.

### RELATED APPLICATIONS

This application claims the benefit of U.S. Provisional Application Nos. 63/344,055, filed May 20, 2022, and 63/464,018, filed May 4, 2023, the entire disclosure of each of which is incorporated herein by reference.

### STATEMENT REGARDING FEDERALLY SPONSORED RESEARCH

This invention was made with Government support under Grant Numbers SC3GM084824 and SC3GM116713, awarded by the National Institutes of Health, and W81XWH-20-1-0041 awarded by the US Army. This work was also supported by the Puerto Rico Science and Technology Research Trust. The Government has certain rights in the invention.

### BACKGROUND

Breast cancer metastasis reduces patient survival rates from 90% to 20%. Metastasis remains the primary cause of mortality from breast cancer due to the heterogeneity of malignant breast cancer and the lack of targeted therapeutics that can prevent and (or) eliminate metastases. Therefore, there is unmet medical need for the translational development of Rac/Cdc42 inhibitors as anti-metastatic cancer therapeutics. The rationale stems from a large body of data directly implicating the Rho GTPases Rac and Cdc42 and their downstream effector p21-activated kinase (PAK) as pivotal regulators of metastatic cancer cell migration and invasion; and thus, metastasis. Cancer cell migration and invasion are essential drivers of metastasis, which involves migration away from the primary tumor. Accordingly, dysregulation of Rho GTPases (Rho, Rac, Cdc42), which are essential for cancer cell migration, are central to metastatic cancer progression. Studies have found that hyperactive Rac1 and Rac3 causally increase survival, proliferation, and invasion of many cancer types. In addition to promoting cancer malignancy, Rac, and the close homolog Cdc42, are essential for Ras and other oncogene-mediated transformation. Cdc42 has also been implicated in transformation, tumorigenesis, and invasion. Accordingly, effective inhibitors of the Rho GTPases Rac and Cdc42 and their downstream effector p21-activated kinase (PAK) are needed.

### SUMMARY

In one aspect, the disclosure relates to a compound of the formula I, or a pharmaceutically acceptable salt thereof,

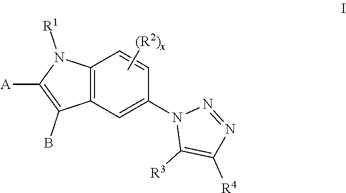

wherein A and B are independently H, deuterium, halogen, alkyl, alkenyl, alkynyl, cycloalkyl, heterocycloalkyl, aryl, or heteroaryl, —OH, —CN, amino, amide, alkoxy, carboxy, ester, thioether, sulfoxide, sulfone, sulfonamide; or A and B taken together with the atom to which they are attached form cycloalkyl, aryl, heterocycloalkyl, or heteroaryl;
R¹ is H, deuterium, alkyl, alkenyl, alkynyl, cycloalkyl, heterocycloalkyl, aryl, or heteroaryl;
each R₂ is independently deuterium, halogen, alkyl, alkenyl, alkynyl, cycloalkyl, aryl, —OH, —CN, amino, amide, alkoxy, carboxy, ester, thioether, sulfoxide, sulfone, or sulfonamide;
R³ is halogen, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, carboxy, ester, thioether, sulfoxide, sulfone, or sulfonamide;
R⁴ is halogen, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, carboxy, ester, thioether, sulfoxide, sulfone, or sulfonamide;
x is 0, 1, 2, or 3.

In certain embodiments of the above aspects, the compound of Formula I, Ia, Ib, Ic, and Id is a compound selected from those species described or exemplified in the detailed description below.

In further aspects, the disclosure relates to a pharmaceutical composition comprising at least one compound of Formula I, Ia, Ib, Ic, and Id, or a pharmaceutically acceptable salt thereof. Pharmaceutical compositions according to the disclosure may further comprise a pharmaceutically acceptable excipient.

In further aspects, the disclosure relates to a compound of Formula I, Ia, Ib, Ic, and Id, or a pharmaceutically acceptable salt thereof, for use as a medicament.

In further aspects, the disclosure relates to a method of treating disease, such as cancer comprising administering to a subject in need of such treatment an effective amount of at least one compound of Formula I, Ia, Ib, Ic, and Id, or a pharmaceutically acceptable salt thereof.

In further aspects, the disclosure relates to a use of a compound of Formula I, Ia, Ib, Ic, and Id, or a pharmaceutically acceptable salt thereof, in the preparation of a medicament for the treatment of disease, such as cancer, and the use of such compounds and salts for treatment of such diseases.

In further aspects, the disclosure relates to a method of treating cancer comprising contacting a cell with an effective amount of at least one compound of Formula I, Ia, Ib, Ic, and Id, or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

### BRIEF DESCRIPTION OF THE DRAWINGS

FIG. 1A shows bar graphs of the effect of compounds according to the present disclosure on viability of MDA-MB-231, MDA-MB-435, and MDA-MB-468 breast cancer cell lines. Cells plated at equal density were treated at 500 nM of compounds. After 72 hours, cell viability was measured by a MTT assay. n=3; * p<0.05, ** p<0.01, *** p<0.001.

FIG. 1B shows bar graphs of the effect of compounds according to the present disclosure on caspase-3/7 activities for MDA-MB-231, MDA-MB-435, and MDA-MB-468 breast cancer cell lines. Cells plated at equal density were treated at 500 nM of compounds. After 48 hours, caspase 3/7 activity was measured by luminescence. n=3; * p<0.05, ** p<0.01.

FIG. 1C shows a bar graph of the effect of compound 4c on viability of MDA-MB-231, MDA-MB-435, MCF7, or MCF-10 cell lines. Cells plated at equal density were treated at 250 nM of 4c at 250 nM.

FIG. 2A shows a graph of GIso curves for percentage cell viability relative to vehicle of MDA-MB-231, MDA-MB-435, and MCF-10A cells treated for 120 hours with 4c at concentrations ranging from 0-1000 nM. Data from 3 biologic replicates each with 2 technical replicates. Four parameter dose-response curves generated using GraphPad Prism are shown.

FIG. 2B shows a graph of the effect of 4c on viability of human mammary epithetial cells (HMEC) cell line. Cells were treated with 4c at concentrations ranging from 0-1000 nM. Cell viability was assessed by a MTT assay. n=2.

FIG. 3A shows micrographs of the effect of 4c on cell migration in a wound healing assay. MDA-MB-231 cells plated at equal density were subjected to a scratch in the center and treated with 4c at 0 or 250 nM.

FIG. 3B shows a bar graph of the percentage of wound closure relative to vehicle (100%). Mircrographs were digitally acquired at 0, 12, and 24 hours and the area of the wound quantified relative to the distance at time 0. n=3; * p<0.05, **** p<0.0001.

FIG. 3C shows representative fluorescent micrographs of MDA-MB-231 breast cancer cells following vehicle or 4c treatment. Cells were treated with 0 or 250 nM 4c for 12 hours and fixed and stained with Rhodamine Phalloidin.

FIG. 4A shows representative fluorescent micrographs of A549 lung adenocarcinoma cells that were serum-starved for 24 hours, and treated with vehicle or 250, 500, or 1000 nM 4c for 24 hours. Cells were stimulated with EGF (200 ng/ml) for 5 min, and fixed and stained with Rhodamine Phalloidin for F-actin.

FIG. 4B shows a bar graph of ruffle area/cell quantified in the A549 lung adenocarcinoma cell line from 5 microscopic fields/slide, n=3.

FIG. 5A shows an image of the Western blot for cell lysates. MDA-MB-231 breast cancer cells were treated for 24 hours with 0 or 250 nM 4c. Cells were lysed and equal amounts of proteins were subjected to pull-down assays using the p21-binding domain of PAK to isolate GTP bound Rac. Top panel, pulldown showing GTP bound Rac1; middle panel, total Rac1 in lysates; bottom panel, actin as a loading control.

FIG. 5B shows a bar graph of the integrated density for active Rac1 divided by total Rac1 relative to vehicle.

FIG. 6A shows an image of the Western blot for cell lysates. GST-Rac1 (G15A) beads were pre-incubated with vehicle (0.1% DMSO), or 8 μM EHop-016, or 250 nM Compound 4c for 1 hour followed by incubation with MDA-MB-231 cell lysates (equal total protein). Representative Western blot for Rac1(G15A) or pulldowns immunostained for Vav2 is shown (n=3).

FIG. 6B shows a graph of the observed changes in fluorescence resonance energy transfer (FRET) between the fluorescent mant group of mant-GDP or mant-GTP and the conserved tryptophan 56 of Rac1 monitored by fluorescence (ex 290 nm/em 440 nm) in the presence or absence of purified Vav2 DH/PH domain. 2 μM purified Rac.GDP was incubated in a solution of 20 mM Tris-HCl (pH 7.5), 50 mM NaCl, 1 mM MgCl₂, 2 mM mant-GDP with 0, 2, 10 μM 4c for 4 min. Exchange was then initiated by addition of Vav2 to a final concentration of 5 μM. n=3.

FIG. 7 shows an image of the Western blot for cell lysates for Rac1B. MDA-MB-468 breast cancer cells were treated for 24 hours with 0 or 250 nM MBQ-167 or compound 4c. Cells were lysed and equal amounts of proteins were subjected to pull-down assays using the p21-binding domain of PAK to isolate GTP bound Rac. n=3.

FIG. 8 shows a graph of GI₅₀ curves for percentage cell viability relative to vehicle of Panc-1 human pancreatic cancer cells treated for 120 hours with MBQ-167, MBQ-168, or compound 4c at concentrations ranging from 0-1000 nM. n=3

FIG. 9A shows a bar graph of the percentage of wound closure relative to vehicle (100%). micrographs of the effect of MBQ-167 on cell migration in a wound healing assay. Panc-1 cells plated at equal density were subjected to a scratch in the center and treated with MBQ-167 at 0, 150 nM, 250 nM, or 500 nM. Mircrographs were digitally acquired at 0, 12, and 24 hours and the area of the wound quantified relative to the distance at time 0. n=3.

FIG. 9B shows a bar graph of the percentage of wound closure relative to vehicle (100%). micrographs of the effect of 4c on cell migration in a wound healing assay. Panc-1 cells plated at equal density were subjected to a scratch in the center and treated with 4c at 0, 150 nM, 250 nM, or 500 nM. Mircrographs were digitally acquired at 0, 12, and 24 hours and the area of the wound quantified relative to the distance at time 0. n=3.

FIG. 10A shows a bar graph of the percentage of wound closure relative to vehicle (100%). micrographs of the effect of MBQ-167 on cell migration in a wound healing assay. Murine macrophage cell line, RAW264.7, plated at equal density were subjected to a scratch in the center and treated with MBQ-167 at 0, 150 nM, 250 nM, or 500 nM. Mircrographs were digitally acquired at 0, 12, and 24 hours and the area of the wound quantified relative to the distance at time 0. n=3.

FIG. 10B shows a bar graph of the percentage of wound closure relative to vehicle (100%). micrographs of the effect of 4c on cell migration in a wound healing assay. Murine macrophage cell line, RAW264.7, plated at equal density were subjected to a scratch in the center and treated with 4c at 0, 150 nM, 250 nM, or 500 nM. Mircrographs were digitally acquired at 0, 12, and 24 hours and the area of the wound quantified relative to the distance at time 0. n=3.

FIG. 11 shows a bar graph of the percentage of wound closure relative to vehicle (100%). micrographs of the effect of 4c on cell migration in a wound healing assay. SW-982 synovial cells plated at equal density were subjected to a scratch in the center and treated with 4c at 0, 150 nM, 250 nM, or 500 nM. Mircrographs were digitally acquired at 0, 12, and 24 hours and the area of the wound quantified relative to the distance at time 0. n=3.

FIG. 12 shows a bar graph of the percentage cell viability of MBQ-167 and CPV compounds in TNBC MDA-MB-231 cells. MDA-MB-231 cells were treated with vehicle, or 250 nM of MBQ-167, or CPV compounds 5-9 for 48h and subjected to a MTT assay. Cell viability relative to vehicle (100%) is shown.

FIG. 13A shows GI₅₀ curves for CPV compounds in HER2++ cancer cells. MDA-MB-435 cells were treated with 0-1000 nM MBQ-167 or compound 5 for 72 h and the viability determined relative to vehicle. GI₅₀ was calculated using Graph pad Prism.

FIG. 13B shows GIso curves for CPV compounds in HER2++ cancer cells. MDA-MB-435 cells were treated with 0-1000 nM MBQ-167 or compound 7 for 72 h and the viability determined relative to vehicle. GIso was calculated using Graph pad Prism.

FIG. 13C shows GI₅₀ curves for CPV compounds in HER2++ cancer cells. MDA-MB-435 cells were treated with 0-1000 nM MBQ-167 or compound 6 for 72 h and the viability determined relative to vehicle. GI₅₀ was calculated using Graph pad Prism.

FIG. 13D shows G150 curves for CPV compounds in HER2++ cancer cells. MDA-MB-435 cells were treated with 0-1000 nM MBQ-167 or compound 9 for 72 h and the viability determined relative to vehicle. GIso was calculated using Graph pad Prism.

FIG. 13E shows GI₅₀ curves for CPV compounds in HER2++ cancer cells. MDA-MB-435 cells were treated with 0-1000 nM MBQ-167 or compound 8 for 72 h and the viability determined relative to vehicle. GIso was calculated using Graph pad Prism.

FIG. 14A shows an image of the Western blot for cell lysates showing the effect of C5 (compound 5) at 250 nM compared to MBQ-167. MDA-MB-435 breast cancer cells were treated for 24 hours with MBQ-167 at 250 nM or compound 5 at 250 nM. Cells were lysed and equal amounts of proteins were subjected to pull-down assays using a GST-fusion protein of the Cdc42-rac interactive binding (CRIB) domain of P21-activated kinase (PAK). The pulldowns of Rac.GTP and whole cell lysates were western blotted with a pan Rac1/2/3 antibody. Actin is shown as a control. Results are shown for duplicate experiments.

FIG. 14B shows an image of the Western blot for cell lysates showing Rac activation inhibition by 10-100 nM compound 5 (C5). MDA-MB-435 breast cancer cells were treated for 24 hours with vehicle or compound 5 at 10, 50, or 100 nM. Cells were lysed and equal amounts of proteins were subjected to pull-down assays using a GST-fusion protein of the Cdc42-rac interactive binding (CRIB) domain of P21-activated kinase (PAK). The pulldowns of Rac.GTP and whole cell lysates were western blotted with a pan Rac1/2/3 antibody. Actin is shown as a control.

FIG. 14C shows an image of the Western blot for cell lysates showing Rac activation inhibition of compounds 6-9 at 250 nM compared to MBQ-167. MDA-MB-435 breast cancer cells were treated for 24 hours with MBQ-167 at 250 nM or compounds 6-9 at 250 nM. Cells were lysed and equal amounts of proteins were subjected to pull-down assays using a GST-fusion protein of the Cdc42-rac interactive binding (CRIB) domain of P21-activated kinase (PAK). The pulldowns of Rac.GTP and whole cell lysates were western blotted with a pan Rac1/2/3 antibody. Actin is shown as a control.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*” “A —**“,

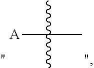

or where each of”—*“, “—**”, and

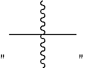

represents a bond to A and the point of covalent bond attachment to B. Alternatively, in some embodiments, the portion of A-B defined by the group or chemical structure B can be represented by “*—B”, “**—B”, or

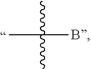

where each of “—*”, “—**”, and

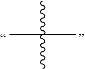

represent a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### PHARMACEUTICAL COMPOSITIONS

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-2microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (or preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with a compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate. alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl) ₂ or —CH₂—OP(O)(O-alkyl) ₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted. As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbyIC (O)—, preferably alkylC (O)—.

The term “acylamino” is art-recognized and refers to an amino group substituted with an acyl group and may be represented, for example, by the formula hydrocarbylC (O) NH—.

The term “acyloxy” is art-recognized and refers to a group represented by the general formula hydrocarbylC (O)O—, preferably alkylC (O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁-6alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

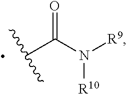

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

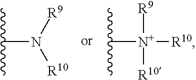

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

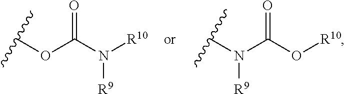

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0] oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbonate” is art-recognized and refers to a group —OCO₂—.

The term “carboxy”, as used herein, refers to a group represented by the formula-CO₂H.

The term “ester”, as used herein, refers to a group —C(O) OR⁸ wherein R⁸ represents a hydrocarbyl group.

The term “ketone”, as used herein, refers to a group —C(O) R⁷ wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a=O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

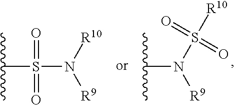

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O) ₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O) SR8 or SC(O) R⁸ wherein R⁸ represents a hydrocarbyl.

The term “thioether”, as used herein, is equivalent to an ether, wherein the oxygen is replaced with a sulfur.

The term “urea” is art-recognized and may be represented by the general formula

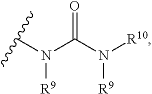

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

As used herein, the term “cancer metastasis inhibitor” as used herein refers to a compound that decreases or prevents the spread of cancer growth and proliferation.

### REPRESENTATIVE EMBODIMENTS

In some embodiments, the disclosure relates to a compound of the formula I, or a pharmaceutically acceptable salt thereof,

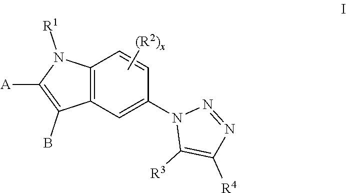

wherein A, B, R¹, R², R³, R⁴, R⁵, and x are as described herein.

In certain embodiments, the compounds have the structural formula (Ia),

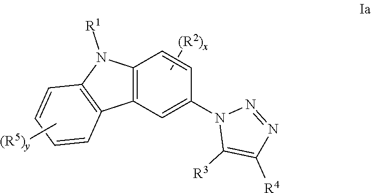

wherein R¹, R², R³, R⁴, R⁵, x, and y are as described herein.

In certain embodiments, the compounds have the structural formula (Ib),

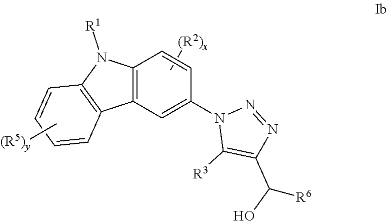

wherein R¹, R², R³, R⁵, R⁶, x, and y are as described herein.

In certain embodiments, the compounds have the structural formula (Ic)

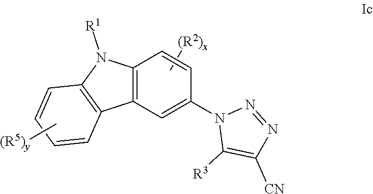

wherein R¹, R², R³, x, and y are as described herein.

In certain embodiments, the compounds have the structural formula (Id)

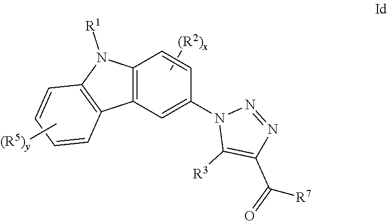

wherein R¹, R², R³, R⁵, R⁷, x, and y are as described herein.

In some embodiments, A and B are independently H, deuterium, halogen, alkyl, alkenyl, alkynyl, cycloalkyl, heterocycloalkyl, aryl, or heteroaryl, —OH, —CN, amino, amide, alkoxy, carboxy, ester, thioether, sulfoxide, sulfone, sulfonamide; or A and B taken together with the atom to which they are attached form cycloalkyl, aryl, heterocycloalkyl, or heteroaryl. In some embodiments, A and B taken together with the atom to which they are attached form cycloalkyl, aryl, heterocycloalkyl, or heteroaryl. In some embodiments, A and B taken together with the atom to which they are attached form aryl. In some preferred embodiments, A and B taken together with the atom to which they are attached form C₆-C₁₀ aryl. In certain preferred embodiments, A and B taken together with the atom to which they are attached form phenyl.

In some embodiments, R¹ is H, deuterium, alkyl, alkenyl, alkynyl, cycloalkyl, heterocycloalkyl, aryl, or heteroaryl. In some embodiments, R¹ is alkyl. In some preferred embodiments, R¹ is C₁-C₆ alkyl. In some embodiments, RI is C₁-C₄ alkyl. In some embodiments, R¹ is methyl, ethyl, propyl, isopropyl, butyl, isobutyl, secbutyl, or tertbutyl. In some embodiments, R¹ is methyl or ethyl. In some embodiments, R¹ is preferably ethyl.

In some embodiments, each R² is independently deuterium, halogen, alkyl, alkenyl, alkynyl, cycloalkyl, aryl, —OH, —CN, amino, amide, alkoxy, carboxy, ester, thioether, sulfoxide, sulfone, or sulfonamide. In some embodiments, R² is deuterium.

In some embodiments, x is 0, 1, 2, or 3. In some preferred embodiments, x is 0.

In some embodiments, R³ halogen, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, carboxy, ester, thioether, sulfoxide, sulfone, or sulfonamide. In some embodiments, R³ is cycloalkyl, heterocycloalkyl, aryl, or heteroaryl. In some embodiments, R³ is aryl. In some preferred embodiments, R³ is C₆-C₁₀ aryl. In some embodiments, R³ is phenyl, which is optionally substituted by R^{a}, wherein each R^{a} is deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide. In some embodiments, R³ is preferably unsubstituted phenyl.

In some embodiments, R⁴ is halogen, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, carboxy, ester, thioether, sulfoxide, sulfone, or sulfonamide. In some embodiments, R⁴ is halogen, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, ester, thioether, sulfoxide, sulfone, or sulfonamide. In some embodiments, R⁴ is aralkyl, hetaralkyl, hydroxyalkyl, —CN, amide, carboxy, or ester. In some embodiments, R⁴ is aralkyl or hetaralkyl. In some embodiments, R⁴ is C₁-C₆ alkyl-aryl or C₁-C₆ alkyl-heteroaryl, wherein C₁-C₆ alkyl is optionally substituted by deuterium, halogen, alkyl, alkenyl, alkynyl, cycloalkyl, heterocycloalkyl, aryl, or heteroaryl, —OH, —CN, nitro, amino, amide, alkoxy, carboxy, ester, thioether, sulfoxide, sulfone, or sulfonamide. In some embodiments, R⁴ is C₁-C₆ alkyl-aryl or C₁-C₆ alkyl-heteroaryl, wherein C₁-C₆ alkyl is optionally substituted by —OH. In some embodiments, R⁴ is methyl-aryl or methyl-heteroaryl, wherein methyl is optionally substituted by —OH. In some preferred embodiments, R⁴ is hydroxymethyl-aryl or hydroxymethyl-heteroaryl. In some embodiments, R⁴ is hydroxyalkyl, which is optionally substituted by R⁶, wherein R⁶ is aryl or heteroaryl. In some embodiments, R⁴ is hydroxymethyl or hydroxyethyl, which is optionally substituted by R⁶, wherein R⁶ is aryl or heteroaryl. In some embodiments, R⁴ is hydroxymethyl, which is optionally substituted by R⁶, wherein R⁶ is aryl or heteroaryl. In some embodiments, R⁴ is selected from the group consisting of

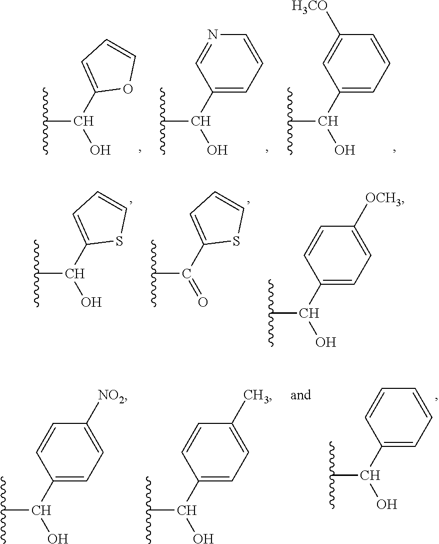

wherein “ [symbol] ” is a point of covalent attachment to triazole ring. In some embodiments, R⁴ is selected from the group consisting of

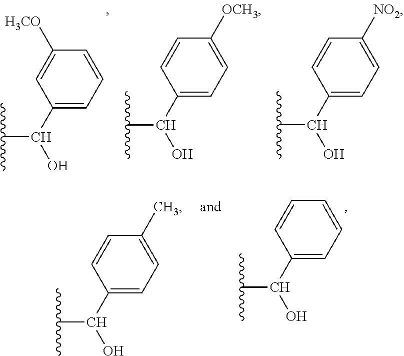

wherein “ [symbol] ” is a point of covalent attachment to triazole ring. In some embodiments, R⁴ is selected from the group consisting of

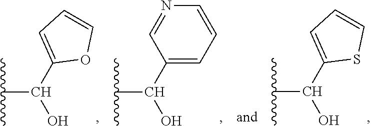

wherein “ [symbol] ” is a point of covalent attachment to triazole ring. In some embodiments, R⁴ is

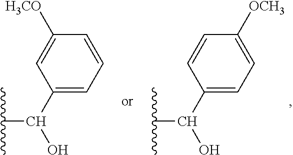 [SMILES: *C(O)c1cccc(OC)c1 ; *C(O)c1ccc(OC)cc1]

wherein “ [symbol] ” is a point of covalent attachment to triazole ring. In some embodiments, R⁴ is

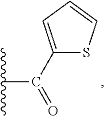 [SMILES: *C(=O)c1cccs1]

wherein “ [symbol] ” is a point of covalent attachment to triazole ring. In some embodiments, R⁴ is preferably

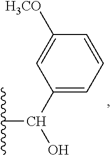 [SMILES: *C(O)c1cccc(OC)c1]

wherein “ [symbol] ” is a point of covalent attachment to triazole ring.

In some preferred embodiments, R⁴ is-CN.

In some embodiments, R⁴ is carboxy.

In some embodiments, R⁴ is ester. In some embodiments, R⁴ is ketone. In some embodiments, R⁴ is amide. In some embodiments, R⁴ is-C(O) OR⁸. In some embodiments, R⁴ is-C(O) R⁷. In some embodiments, R⁴ is-C(O) OR⁸ or —C(O) NR⁹R¹⁰. In some embodiments, R⁴ is-C(O) OR⁸. In some embodiments, R⁴ is-C(O) NR⁹R¹⁰. In some embodiments, R⁴ is selected from the group consisting of

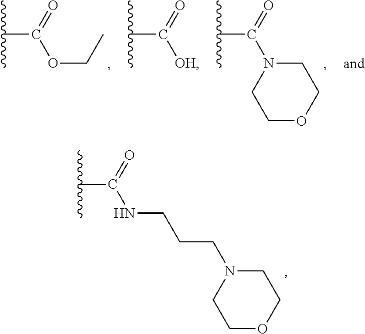

wherein “ [symbol] ” is a point of covalent attachment to triazole ring.

In some embodiments, each R⁵ is independently deuterium, halogen, alkyl, alkenyl, alkynyl, cycloalkyl, aryl, —OH, —CN, nitro, amino, amide, alkoxy, carboxy, ester, thioether, sulfoxide, sulfone, or sulfonamide. In some embodiments, each R⁵ is independently halogen, —OH, —CN, alkyl, alkoxy, or nitro.

In some embodiments, R⁶ is aryl or heteroaryl.

In some embodiments, R⁶ is aryl, which is optionally substituted by R^{b}, wherein each R^{b} is independently deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide. In some embodiments, R⁶ is C₆-C₁₀ aryl. In some preferred embodiments, R⁶ is phenyl, which is optionally substituted by R^{b}. In some embodiments, R⁶ is unsubstituted phenyl. In some embodiments, R⁶ is methylphenyl. In some embodiments, R⁶ is 4-methylphenyl. In some embodiments, R⁶ is preferably methoxyphenyl, such as 3-methoxyphenyl or 4-methoxyphenyl. In certain preferred embodiments, R⁶ is 3-methoxyphenyl. In some embodiments, R⁶ is 4-methoxyphenyl. In some embodiments, R⁶ is nitrophenyl. In some embodiments, R⁶ is 4-nitrophenyl.

In some embodiments, R⁶ is heteroaryl, which is optionally substituted by R^{b}, wherein each R^{b} is independently deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide. In some embodiments, R⁶ is 5- to 8-membered heteroaryl. In some embodiments, R⁶ is 5-membered heteroaryl or 6-membered heteroaryl. In some embodiments. R⁶ is 5-membered heteroaryl (e.g., furan or thiophene). In some embodiments, R⁶ is unsubstituted furan or thiophene. In some embodiments, R⁶ is unsubstituted furan. In some embodiments, R⁶ is unsubstituted thiophene. In some embodiments, R⁶ is 2-furan or 2-thiophene. In some embodiments. R⁶ is 2-furan. In some embodiments, R⁶ is 2-thiophene. In some embodiments, R⁶ is 6-membered heteroaryl. In some embodiments, R⁶ is unsubstituted 6-membered heteroaryl. In some embodiments, R⁶ is pyridine. In some embodiments, R⁶ is 3-pyridine.

In some embodiments, R⁷ is alkyl, aryl, heteroaryl, —OR⁸ or —NR⁹R¹⁰. In some embodiments, R⁷ is aryl or heteroaryl, which is optionally substituted by R^{b}, wherein each R^{b} is independently deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide. In some embodiments, R⁷ is heteroaryl, —OR⁸ or —NR⁹R¹⁰. In some embodiments, R⁷ is heteroaryl (e.g., thienyl), which is optionally substituted by R^{b}, wherein each R^{b} is independently deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide. In some embodiments, R⁷ is 5- to 8-membered heteroaryl. In some embodiments, R⁷ is 5-membered heteroaryl or 6-membered heteroaryl. In some embodiments, R⁷ is 5-membered heteroaryl (e.g., furan or thiophene, preferably thiophene). In some embodiments, R⁷ is unsubstituted furan or thiophene. In some embodiments, R⁷ is unsubstituted thiophene. In some embodiments, R⁷ is 2-furan or 2-thiophene. In some embodiments, R⁷ is 2-furan. In some embodiments, R⁷ is 2-thiophene. In some embodiments, R⁷ is-OR⁸. In some embodiments, R⁷ is-NR⁹R¹⁰. In some embodiments, R⁷ is alkoxy. In some embodiments, R′ is C₁-C₆ alkoxy. In some embodiments, R⁷ is C₁-C₄ alkoxy. In some embodiments, R⁷ is methoxy or ethoxy. In some embodiments, R⁷ is ethoxy. In some embodiments, R⁷ is-NR⁹R¹⁰. In some embodiments, R⁷ is-NHR9. In some embodiments, R⁷ is-NH-heterocyclylalkyl. In some embodiments, R⁷ is-NH—C₁-C₆ alkyl-heterocyclyl. In some embodiments, R⁷ is-NH-propyl-heterocyclyl. In some embodiments, R⁷ is-NH-propyl-3- to 10-membered heterocyclyl. In some embodiments, R⁷ is-NH-propyl-morpholino.

In some embodiments, each R⁸, R⁹, and R¹⁰ is independently H, deuterium, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl.

In some embodiments, R⁸ is H or alkyl. In some embodiments, R⁸ is H. In some embodiments, R⁸ is alkyl. In some embodiments, R³ is C₁-C₆ alkyl. In some embodiments, R⁸ is C₁-C₆ alkyl. In some embodiments, R⁸ is C₁-C₄ alkyl. In some embodiments, R⁸ is methyl, ethyl, propyl, isopropyl, butyl, isobutyl, secbutyl, or tertbutyl. In some embodiments, R⁸ is methyl or ethyl. In some embodiments, R⁸ is ethyl.

In some embodiments, R⁹ is heterocyclylalkyl. In some embodiments, R⁹ is C₁-C₆ alkyl-heterocyclyl. In some embodiments. R⁹ is propyl-(heterocyclyl). In some embodiments, R⁹ is propyl-3- to 10-membered heterocyclyl. In some embodiments, R⁹ is propyl-morpholino. In some embodiments, R¹⁰ is H. In some embodiments, R¹⁰ is H and R⁹ is heterocyclylalkyl.

In some embodiments, R⁹ and R¹⁰ taken together with the atom to which they are attached form heterocycloalkyl or heteroaryl. In some embodiments, R⁹ and R¹⁰ taken together with the atom to which they are attached form heterocycloalkyl. In some embodiments, R⁹ and R¹⁰ taken together with the atom to which they are attached form 3- to 10-membered heterocycloalkyl. In some embodiments, R⁹ and R¹⁰ taken together with the atom to which they are attached form morpholine.

In some embodiments, each R^{a} and R^{b} are independently deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide. In some embodiments, each R^{a} and R^{b} are independently halogen, —OH, —CN, alkyl, alkoxy, or nitro. In some embodiments, each R^{a} and R^{b} are independently alkyl, alkoxy, or nitro. In some embodiments, between 0 and 5 R^{a} are present. In some embodiments, at least one R^{a} is present. In some embodiments, 0 or 1 R^{a} is present. In some embodiments, R^{a} is not present. In some embodiments, between 0 and 5 R^{b} are present. In some embodiments, at least one R^{b} is present. In some embodiments, 0 or 1 R^{b} is present. In some embodiments, R^{b} is not present. In some embodiments, R^{b} is alkyl. In some embodiments, R^{b} is C₁-C₆ alkyl. In some embodiments, R^{b} is C₁-C₆ alkyl. In some embodiments, R^{b} is C₁-C₄ alkyl. In some embodiments, R^{b} is methyl, ethyl, propyl, isopropyl, butyl, isobutyl, secbutyl, or tertbutyl. In some embodiments, R^{b} is methyl or ethyl. In some embodiments, R^{b} is methyl. In some embodiments, R^{b} is alkoxy. In some embodiments, R^{b} is C₁-C₆ alkoxy. In some embodiments, R^{b} is C₁-C₄ alkoxy. In some embodiments, R^{b} is methoxy or ethoxy. In some embodiments, R^{b} is methoxy. In some embodiments, R^{b} is nitro.

In some embodiments, x is 0, 1, 2, or 3. In some embodiments, x is 0 or 1. In some embodiments, x is preferably 0.

In some embodiments, y is 0, 1, 2, 3, or 4. In some embodiments, y is 0 or 1. In some embodiments, y is preferably 0.

In some preferred embodiments, x is 0, y is 0, and R¹ is ethyl. In some embodiments, x is 0, y is 0, and R³ is phenyl. In some preferred embodiments, x is 0, y is 0, R¹ is ethyl, and R³ is phenyl. In some embodiments, x is 0, y is 0, and R¹ is ethyl. In some embodiments, x is 0, y is 0, and R³ is phenyl. In some embodiments, x is 0, y is 0, R¹ is ethyl, R³ is phenyl, and R⁴ is-CN. In some embodiments, x is 0, y is 0, R¹ is ethyl, R³ is phenyl, and R⁴ is hydroxymethyl-aryl.

In some embodiments, the compound of Formula (I) is selected from the group consisting of

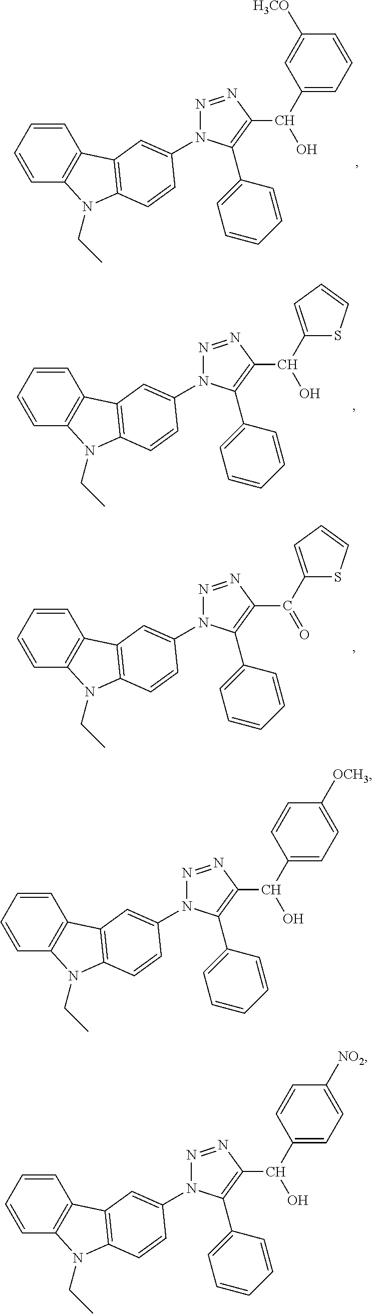 [SMILES: CCn1c2ccccc2c2cc(-n3nnc(C(O)c4cccc(OC)c4)c3-c3ccccc3)ccc21 ; CCn1c2ccccc2c2cc(-n3nnc(C(O)c4cccs4)c3-c3ccccc3)ccc21 ; CCn1c2ccccc2c2cc(-n3nnc(C(=O)c4cccs4)c3-c3ccccc3)ccc21 ; CCn1c2ccccc2c2cc(-n3nnc(C(O)c4ccc(OC)cc4)c3-c3ccccc3)ccc21 ; CCn1c2ccccc2c2cc(-n3nnc(C(O)c4ccc([N+](=O)[O-])cc4)c3-c3ccccc3)ccc21]

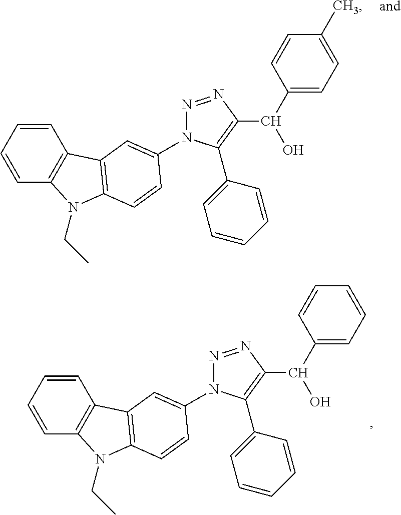 [SMILES: CCn1c2ccccc2c2cc(-n3nnc(C(O)c4ccc(C)cc4)c3-c3ccccc3)ccc21 ; "and" ; CCn1c2ccccc2c2cc(-n3nnc(C(O)c4ccccc4)c3-c3ccccc3)ccc21]

or a pharmaceutically acceptable salt thereof. In some embodiments, the compound of Formula (I) is selected from the group consisting of

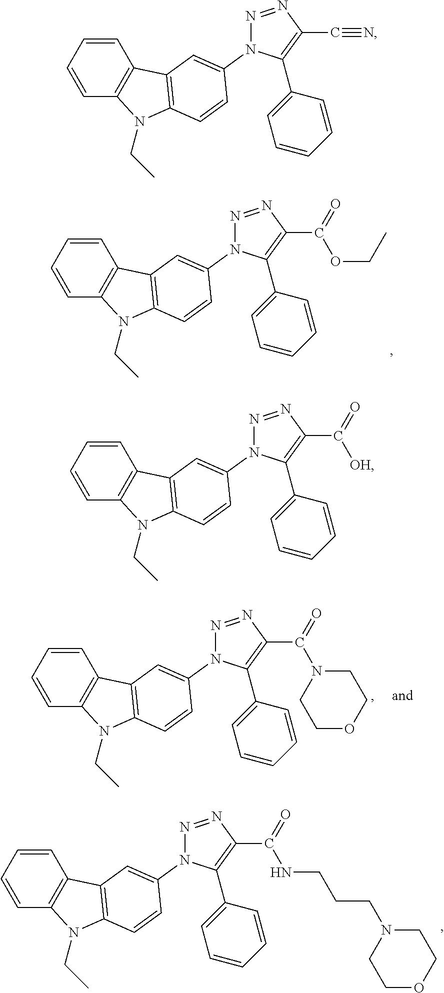 [SMILES: CCn1c2ccccc2c2cc(-n3nnc(C#N)c3-c3ccccc3)ccc21 ; CCOC(=O)c1nnn(-c2ccc3c(c2)c2ccccc2n3CC)c1-c1ccccc1 ; CCn1c2ccccc2c2cc(-n3nnc(C(=O)O)c3-c3ccccc3)ccc21 ; CCn1c2ccccc2c2cc(-n3nnc(C(=O)N4CCOCC4)c3-c3ccccc3)ccc21 ; CCn1c2ccccc2c2cc(-n3nnc(C(=O)NCCCN4CCOCC4)c3-c3ccccc3)ccc21]

or a pharmaceutically acceptable salt thereof. In some embodiments, the compound of Formula (I) is

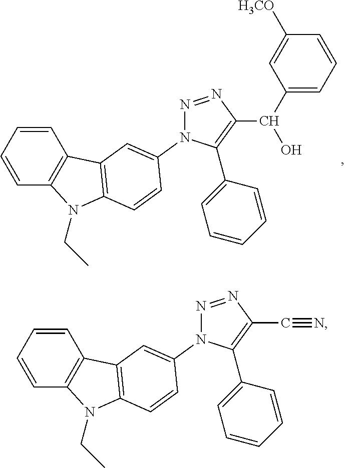 [SMILES: CCn1c2ccccc2c2cc(-n3nnc(C(O)c4cccc(OC)c4)c3-c3ccccc3)ccc21 ; CCn1c2ccccc2c2cc(-n3nnc(C#N)c3-c3ccccc3)ccc21]

or a pharmaceutically acceptable salt thereof. In some embodiments, the compound of Formula (I) is

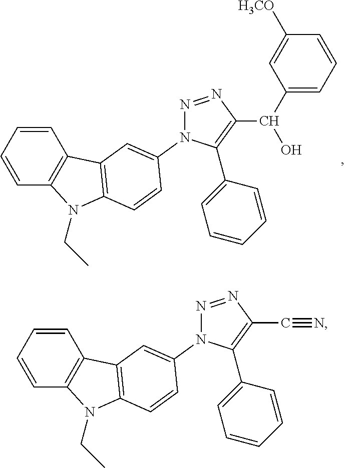 [SMILES: CCn1c2ccccc2c2cc(-n3nnc(C(O)c4cccc(OC)c4)c3-c3ccccc3)ccc21 ; CCn1c2ccccc2c2cc(-n3nnc(C#N)c3-c3ccccc3)ccc21]

or a pharmaceutically acceptable salt thereof.

The following represent illustrative embodiments of compounds of Formula (I):

| Ex. # | Structure | Name |
|---|---|---|
| 4a | 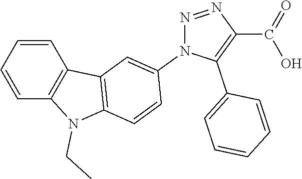 [SMILES: CCn1c2ccccc2c2cc(-n3nnc(C(=O)O)c3-c3ccccc3)ccc21] | (1-(9-ethyl-9H-carbazol-3-yl)-5-phenyl- 1H-1,2,3-triazol-4-yl)(furan-2-yl)methanol |
| 4b | 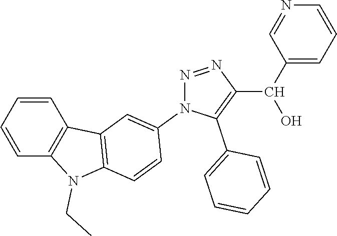 [SMILES: CCn1c2ccccc2c2cc(-n3nnc(C(O)c4cccnc4)c3-c3ccccc3)ccc21] | (1-(9-ethyl-9H-carbazol-3-yl)-5-phenyl- 1H-1,2,3-triazol-4-yl)(pyridin-3- yl)methanol |
| 4c | 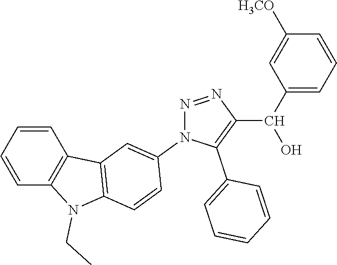 [SMILES: CCn1c2ccccc2c2cc(-n3nnc(C(O)c4cccc(OC)c4)c3-c3ccccc3)ccc21] | (1-(9-ethyl-9H-carbazol-3-y1)-5-phenyl- 1H-1,2,3-triazol-4-yl)(3- methoxyphenyl)methanol |
| 4d | 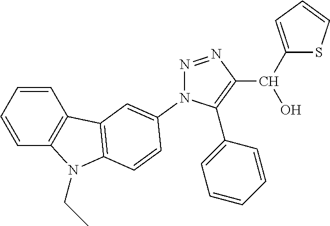 [SMILES: CCn1c2ccccc2c2cc(-n3nnc(C(O)c4cccs4)c3-c3ccccc3)ccc21] | (1-(9-ethyl-9H-carbazol-3-y1)-5-phenyl- 1H-1,2,3-triazol-4-y1)(thiophen-2- yl)methanol |
| 4e | 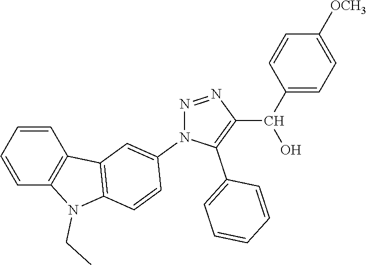 [SMILES: CCn1c2ccccc2c2cc(-n3nnc(C(O)c4ccc(OC)cc4)c3-c3ccccc3)ccc21] | (1-(9-ethyl-9H-carbazol-3-yl)-5-phenyl- 1H-1,2,3-triazol-4-yl)(4- methoxyphenyl)methanol |
| 4f | 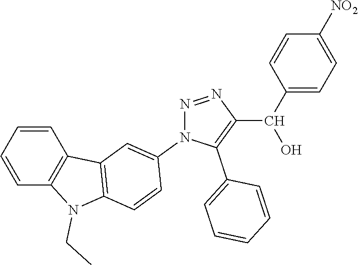 [SMILES: CCn1c2ccccc2c2cc(-n3nnc(C(O)c4ccc([N+](=O)[O-])cc4)c3-c3ccccc3)ccc21] | (1-(9-ethyl-9H-carbazol-3-yl)-5-phenyl- 1H-1,2,3-triazol-4-yl)(4- nitrophenyl)methanol |
| 4g | 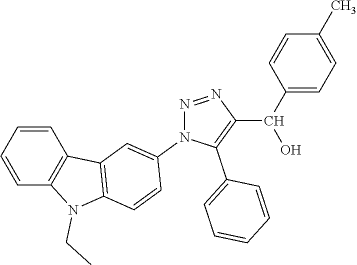 [SMILES: CCn1c2ccccc2c2cc(-n3nnc(C(O)c4ccc(C)cc4)c3-c3ccccc3)ccc21] | (1-(9-ethyl-9H-carbazol-3-yl)-5-phenyl- 1H-1,2,3-triazol-4-y1)(p-toly1)methanol |
| 4h | 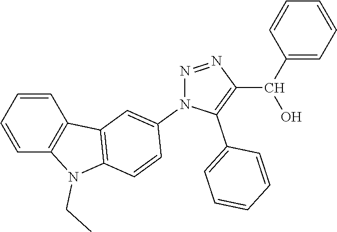 [SMILES: CCn1c2ccccc2c2cc(-n3nnc(C(O)c4ccccc4)c3-c3ccccc3)ccc21] | (1-(9-ethyl-9H-carbazol-3-yl)-5-phenyl- 1H-1,2,3-triazol-4-yl)(phenyl)methanol |
| 5 | 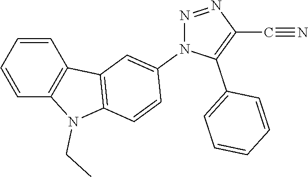 [SMILES: CCn1c2ccccc2c2cc(-n3nnc(C#N)c3-c3ccccc3)ccc21] | 1-(9-ethyl-9H-carbazol-3-yl)-5-phenyl-1H- 1,2,3-triazole-4-carbonitrile |
| 6 | 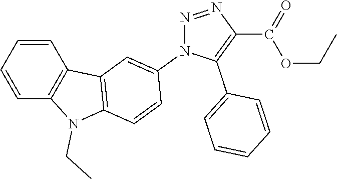 [SMILES: CCOC(=O)c1nnn(-c2ccc3c(c2)c2ccccc2n3CC)c1-c1ccccc1] | Ethyl 1-(9-ethyl-9H-carbazol-3-y1)-5- phenyl-1H-1,2,3-triazole-4-carboxylate |
| 7 | 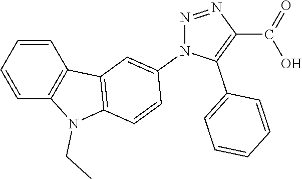 [SMILES: CCn1c2ccccc2c2cc(-n3nnc(C(=O)O)c3-c3ccccc3)ccc21] | 1-(9-Ethyl-9H-carbazol-3-yl)-5-phenyl- 1H-1,2,3-triazole-4-carboxylic acid |
| 8 | 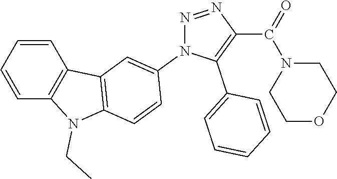 [SMILES: CCn1c2ccccc2c2cc(-n3nnc(C(=O)N4CCOCC4)c3-c3ccccc3)ccc21] | (1-(9-ethyl-9H-carbazol-3-yl)-5-phenyl- 1H-1,2,3-triazol-4- yl)(morpholino)methanone |
| 9 | 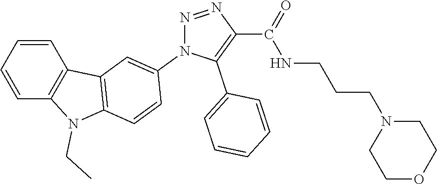 [SMILES: CCn1c2ccccc2c2cc(-n3nnc(C(=O)NCCCN4CCOCC4)c3-c3ccccc3)ccc21] | 1-(9-ethyl-9H-carbazol-3-yl)-N-(3- morpholinopropyl)-5-phenyl-1H-1,2,3- triazole-4-carboxamide |
| 10 | 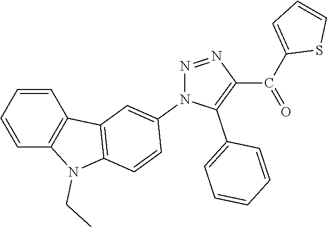 [SMILES: CCn1c2ccccc2c2cc(-n3nnc(C(=O)c4cccs4)c3-c3ccccc3)ccc21] | (1-(9-ethyl-9H-carbazol-3-yl)-5-phenyl- 1H-1,2,3-triazol-4-y1)(thiophen-2- yl)methanone |

and pharmaceutically acceptable salts thereof.

Those skilled in the art will recognize that the species listed or illustrated herein are not exhaustive, and that additional species within the scope of these defined terms may also be selected.

The compounds described herein are useful, for example, as cancer therapeutics, in particular as Rac/Cdc42 inhibitors as anti-metastatic cancer therapeutics. In certain aspects, the present disclosure provides methods of treating breast cancer, lung cancer, prostate cancer, pancreatic cancer, ovarian cancer, gastric cancer, and neuronal cancer. In certain aspects, the present disclosure provides methods of treating breast cancer, for example, triple negative breast cancer (TNBC) or HER2-type breast cancer. In certain aspects, the present disclosure provides methods of treating proliferative diseases, such as breast cancer. In certain aspects, the present disclosure provides methods of inhibiting Rac/Cdc42. In certain aspects, the present disclosure provides methods of inhibiting Rho GTPases Rac and Cdc42 and their downstream effector p21-activated kinase (PAK). In certain aspects, the present disclosure provides methods of inhibiting cancer cell migration. In certain aspects, the present disclosure provides methods of inhibiting metastasis. In certain aspects, the present disclosure provides methods of inhibiting mammosphere formation. In certain aspects, the present disclosure provides methods of inhibiting metastatic cancer progression.

In certain aspects, the present disclosure provides methods of treating an autoimmune disease. In certain aspects, the autoimmune disease is lupus, arthritis, multiple sclerosis, systemic sclerosis, type 1 diabetes, or inflammatory bowel disease.

In certain aspects, the compounds of the present disclosure are for use in treating proliferative diseases, such as breast cancer. In certain aspects, the compounds of the present disclosure are for use in treating breast cancer, lung cancer, prostate cancer, pancreatic cancer, ovarian cancer, gastric cancer, and neuronal cancer. In certain aspects, the compounds of the present disclosure are for use in treating breast cancer, for example, triple negative breast cancer (TNBC) or HER2-type breast cancer. In certain aspects, the compounds of the present disclosure are for use in inhibiting Rac/Cdc42. In certain aspects, the compounds of the present disclosure are for use in inhibiting Rho GTPases Rac and Cdc42 and their downstream effector p21-activated kinase (PAK). In certain aspects, the compounds of the present disclosure are for use in inhibiting cancer cell migration. In certain aspects, the compounds of the present disclosure are for use in inhibiting metastasis. In certain aspects, the compounds of the present disclosure are for use in inhibiting mammosphere formation. In certain aspects, the compounds of the present disclosure are for use in inhibiting metastatic cancer progression. In certain aspects, the compounds of the present disclosure are for use in inducing cell cycle arrest of a diseased cell. In certain aspects, the compounds of the present disclosure are for use in inducing apoptosis of a diseased cell.

In certain aspects, the compounds of the present disclosure are for use in treating a mammal suffering from cancer. In certain embodiments, the cancer is breast cancer. In certain embodiments, the cancer is triple negative breast cancer (TNBC). In certain embodiments, the cancer is HER2-type breast cancer. In certain embodiments, the cancer is metastatic. In certain embodiments, the cancer is non-metastatic.

In certain aspects, the compounds of the present disclosure are for use in treating a mammal suffering from an autoimmune disease. In certain aspects, the autoimmune disease is lupus, arthritis, multiple sclerosis, systemic sclerosis, type 1 diabetes, or inflammatory bowel disease.

In certain aspects, the present disclosure provides methods of inhibiting a GTPase, comprising contacting the GTPase with a compound or composition of the disclosure. In certain embodiments, the GTPase is Rac. In certain embodiments, the GTPase is Cdc42.

In certain aspects, the present disclosure provides methods of treating a mammal suffering from cancer, comprising administering a compound or composition of the disclosure.

In certain embodiments, the cancer is breast cancer. In certain embodiments, the cancer is breast cancer. In certain embodiments, the cancer is triple negative breast cancer (TNBC). In certain embodiments, the cancer is HER2-type breast cancer. In certain embodiments, the cancer is metastatic. In certain embodiments, the cancer is non-metastatic.

In certain aspects, the present disclosure provides methods of treating a mammal suffering from autoimmune diseases such as lupus, arthritis, multiple sclerosis, systemic sclerosis, type 1 diabetes, and inflammatory bowel disease, comprising administering a compound or composition of the disclosure.

In certain embodiments, compounds of the invention are prodrugs of the compounds described herein. For example, wherein a hydroxyl in the parent compound is presented as an ester or a carbonate, or a carboxylic acid present in the parent compound is presented as an ester. In certain such embodiments, the prodrug is metabolized to the active parent compound in vivo (e.g., the ester is hydrolyzed to the corresponding hydroxyl or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomers. For example, a compound of the invention may have greater than 30% de, 40% de, 50% de, 60% de, 70% de, 80% de, 90% de, or even 95% or greater de.

In certain embodiments, the present invention provides pharmaceutical compositions comprising a compound described herein, such as a compound of Formula I, Ia, Ib, Ic, or Id. In certain embodiments, the pharmaceutical compositions further comprise a pharmaceutically acceptable excipient.

In certain embodiments, the pharmaceutical compositions may be for use in treating or preventing a condition or disease as described herein.

In certain embodiments, the compound is Formula (I)

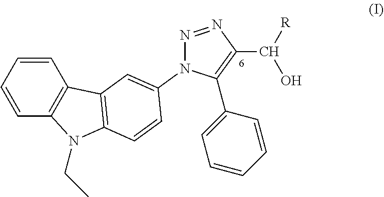

in which R is a substituted aromatic or heteroaromatic group.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula I, Ia, Ib, Ic, and Id.

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| CDCl₃ | deuterated chloroform |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| ESI | Electrospray ionization |
| m/z | mass-to-charge ratio |
| Ms | methanesulfonyl |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| DIAD | diisopropyl azodicarboxylate |
| DMSO | dimethyl sulfoxide |
| MeTHF | 2-methyltetrahydrofuran |
| MOM | methoxymethyl |
| DCM | dichloromethane |
| DMF | N,N-dimethylformamide |
| DBU | 1,8-diazabicyclo[5.4.0]undec-7-ene |
| DIPEA | N,N-diisopropylethylamine |
| Hex | hexanes |
| Pd(dppf)Cl₂ | [1,1′- |
| | Bis(diphenylphosphino)ferrocene]dichloropalladium(II) |
| MeCN (ACN) | Acetonitrile |
| Hunig's Base | N,N-diisopropylethylamine |
| TBAF | Tert butyl ammonium fluoride |
| HBTU | (2-(1H-benzotriazol-1-yl)-1,1,3,3-tetramethyluronium |
| | hexafluorophosphate |
| PPh₃ | Triphenyl phosphine |
| RT | Room Temperature |
| p-TSA | Para-Tolylsulfonic acid |
| t-BuOH | Tert-Butanol |
| mCPBA | Meta-Chloroperoxy benzoic acid |
| AcOH | Acetic Acid |
| DMAc | N,N-Dimethylformamide |

The proposed targets can be prepared via the conventional chemistry or following the general schemes as shown below.

### General Procedure for 4-(Aryl)Methanol Derivatives, 4a-4h

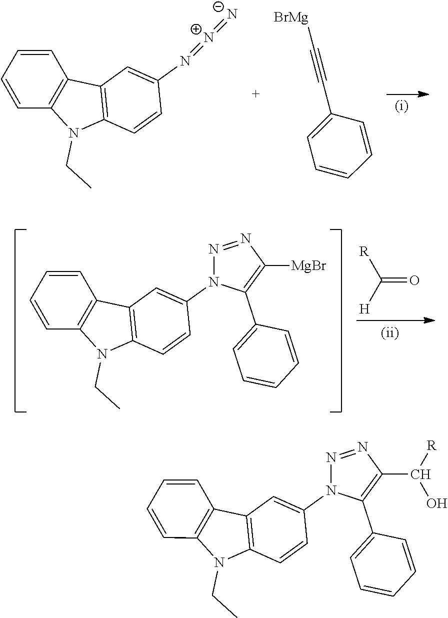

In a 25-mL three-neck round-bottom flask containing phenylacetylene (1.0 mmol) under a nitrogen atmosphere, a solution of 1.0 M (phenylethynyl) magnesium bromide in THF (1.1 mmol) was added drop-wise at 25° C. After the Grignard reagent was added, the mixture was heated at 50° C. for 15 minutes and cooled to 25° C. Neat azide (3-azido-9-ethyl-9H-carbazole) (0.8 mmol) was added and the reaction mixture heated to 50° C. for 1 hour. After addition of the corresponding aldehydes (1.0 mmol, neat) and stirring for 1 hour at room temperature, a solution of 10% ammonium chloride is added and the products extracted with ethyl acetate (3×). The organic layer was washed with brine (3×), separated and dried on sodium sulfate, filtered and concentrated on a rotary evaporator to obtain the crude material. The crude oil was purified by silica gel flash column chromatography to afford the corresponding 1,4,5-trisubstituted-1,2,3-triazole.

### (1-(9-ethyl-9H-carbazol-3-yl)-5-phenyl-1H-1,2,3-triazol-4-yl) (furan-2-yl) methanol (4a)

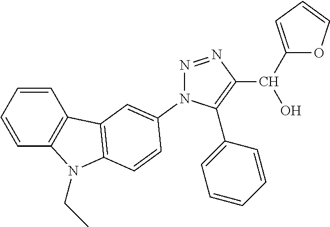 [SMILES: CCn1c2ccccc2c2cc(-n3nnc(C(O)c4ccco4)c3-c3ccccc3)ccc21]

Brown solid (0.1 mmol, 13%, 0.0437 g); Rf=0.52 (Hexane/Ethyl Acetate, 1:1); 1H-NMR (CDCl₃, 500 MHz) δ 1.46 (t, J=7.2 Hz, 3H), 1.59 (s, 1H), 2.06 (s, 1H), 4.39 (q, J=7.2 Hz, 2H), 5.93 (d, J=6.4 Hz, 1H), 6.44-6.35 (m, 5H), 7.40-7.16 (m, 5H), 7.44 (dd, J=8.3, 4.6 Hz, 1H), 7.52 (dd, J=11.3, 4.1 Hz, 1H), 8.00 (d, J=7.8 Hz, 1H), 8.11 (d, J=1.8 Hz, 1H) ppm; ¹³C (CDCl₃, 100 MHz) δ 13.79, 37.81, 62.74, 108.00, 108.31, 108.56, 108.91, 109.02, 110.44, 117.70, 119.53, 120.85, 122.46, 122.78, 123.06, 126.54, 126.62, 128.07, 128.73, 129.34, 129.82, 139.65, 140.63, 142.57, 154.50 ppm. FTMS (ESI) m/z calcd for C₂₇H₂₂N₄O₂, [M+H]⁺435.1822, found 435.1796.

(1-(9-ethyl-9H-carbazol-3-yl)-5-phenyl-1H-1,2,3-triazol-4-yl) (pyridin-3-yl) methanol (4b)

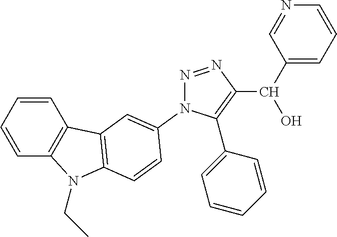 [SMILES: CCn1c2ccccc2c2cc(-n3nnc(C(O)c4cccnc4)c3-c3ccccc3)ccc21]

Bone-white solid (0.13 mmol, 16%, 0.0555 g); Rf=0.26 (Hexane/Ethyl Acetate, 1:1); 1H-NMR (CDCl₃, 500 MHz) δ 1.46 (t, J=7.2 Hz, 3H), 2.07 (s, 1H), 4.38 (q, J=7.2 Hz, 2H), 6.06 (s, 1H), 7.18 (d, J=7.1 Hz, 1H), 7.39-7.23 (m, 4H), 7.45 (d, J=8.2 Hz, 2H), 7.53 (t, J=7.7 Hz, 2H), 7.96 (dd, J=18.8, 7.9 Hz, 4H), 8.08 (d, J=1.9 Hz, 1H), 8.53 (d, J=4.0 Hz, 1H), 8.62 (s, 1H) ppm; ¹³C (CDCl₃, 100 MHz) δ 13.88, 37.88, 66.76, 108.68, 109.00, 117.70, 119.64, 120.81, 122.50, 122.76, 123.12, 123.56, 126.55, 126.74, 127.99, 128.92, 129.50, 129.96, 135.02, 135.40, 139.74, 140.65, 140.71, 146.24, 148.10, 148.68 ppm. FTMS (ESI) m/z calcd for C₂₈H₂₃N₅O, [M+H]⁺446.1982, found 446.1954.

### (1-(9-ethyl-9H-carbazol-3-yl)-5-phenyl-1H-1,2,3-triazol-4-yl) (3-methoxyphenyl) methanol, (4c)

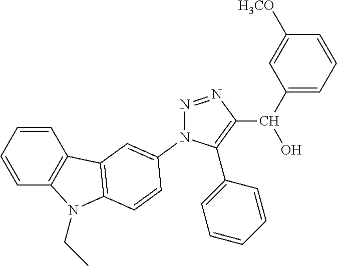 [SMILES: CCn1c2ccccc2c2cc(-n3nnc(C(O)c4cccc(OC)c4)c3-c3ccccc3)ccc21]

Light-brown solid (0.026 mmol, 3.3%, 0.0123 g); R_{f}=0.49 (Hexane/Ethyl Acetate, 1:1); 1H-NMR (CDCl₃, 500 MHz) δ 1.29 (s, 2H), 1.46 (t, J=7.2 Hz, 3H), 4.38 (q, J=7.2 Hz, 2H), 5.93 (s, 1H), 6.95-6.86 (m, 2H), 7.20-7.14 (m, 2H), 7.39-7.22 (m, 6H), 7.45 (d, J=8.5 Hz, 3H), 7.57-7.49 (m, 3H), 7.99 (d, J=7.7 Hz, 1H), 8.22-8.06 (m, 1H) ppm; ¹³C (CDCl₃, 100 MHz) δ 13.76, 37.76, 55.27, 68.34, 108.52, 108.86, 113.83, 117.60, 119.46, 120.69, 122.43, 122.73, 122.97, 126.56, 126.83, 128.09, 128.22, 128.61, 129.07, 129.91, 132.22, 134.66, 134.71, 134.77, 139.56, 140.58, 147.09, 159.14 ppm. FTMS (ESI) m/z calcd for C₃₀H₂₆N₄O₂, [M+H]⁺475.2135, found 475.2106.

### (1-(9-ethyl-9H-carbazol-3-yl)-5-phenyl-1H-1,2,3-triazol-4-yl) (thiophen-2-yl) methanol, (4d)

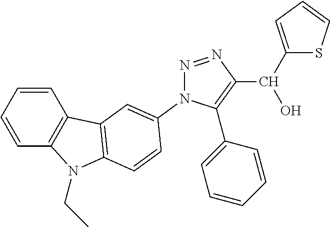 [SMILES: CCn1c2ccccc2c2cc(-n3nnc(C(O)c4cccs4)c3-c3ccccc3)ccc21]

Brown solid (0.2 mmol, 29%, 0.1059 g); R_{f}=0.57 (Hexane/Ethyl Acetate, 1:1); 1H-NMR (CDCl₃, 500 MHz) δ 1.44 (t, J=7.2 Hz, 3H), 2.04 (s, 1H), 4.36 (q, J=7.2 Hz, 2H), 6.13 (d, J=3.0 Hz, 1H), 6.96 (dd, J=1.4, 3.6 Hz, 1H), 7.03 (d, J=3.5, 1H), 7.19-7.34 (m, 9H), 7.42 (d, J=8.2, 1H), 7.51 (s, 1H), 7.97 (d, J=7.8, 1H), 8.08 (d, J=1.8, 1H). ¹³C (CDCl₃, 100 MHz) δ 13.79, 37.81, 64.81, 108.58, 108.90, 117.67, 119.53, 120.74, 122.45, 122.77, 123.05, 125.60, 125.81, 126.61, 126.61, 126.71, 128.06, 128.76, 129.25, 129.83, 134.78, 139.65, 140.63, 146.29, 146.38; FTMS (ESI) m/z calculated for C₂₇H₂₂NOS, [M+H]⁺451.1587, 451.1578.

### (1-(9-ethyl-9H-carbazol-3-yl)-5-phenyl-1H-1,2,3-triazol-4-yl) (4-methoxyphenyl) methanol (4e)

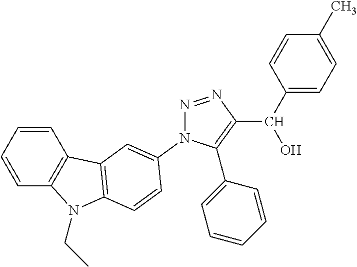 [SMILES: CCn1c2ccccc2c2cc(-n3nnc(C(O)c4ccc(C)cc4)c3-c3ccccc3)ccc21]

Light brown solid (0.25 mmol, 38%, 119.16 mg). R_{f}=0.57 (Hexane/Ethyl Acetate, 1:1); 1H-NMR (CDCl₃, 500 MHz) δ 1.44 (t, J=7.2 Hz, 3H), 2.06 (s, 1H), 3.82 (s, 3H), 4.36 (q, J=7.2 Hz, 2H), 5.91 (d, J=5.5 Hz, 1H), 6.89 (d, J=8.6 Hz, 2H), 7.15 (d, J=7.2 Hz, 1H), 7.23-7.34 (m, 5H), 7.44 (d, J=8.4 Hz, 4H), 7.51 (t, J=7.6 Hz, 1H), 7.97 (d, J=7.8 Hz, 2H), 8.07 (d, J=1.5 Hz, 1H); ¹³C (CDCl₃, 100 MHZ) δ 13.87, 37.87, 55.39, 68.52, 76.85, 77.11, 77.36, 108.63, 108.97, 113.95, 117.72, 119.57, 120.80, 122.55, 122.85, 123.09, 126.66, 126.99, 128.24, 128.35, 128.72, 129.16, 130.01, 134.72, 134.84, 139.67, 140.70, 147.18, 159.28; FTMS (ESI) m/z calculated for C₃₀H₂₆N₄O₂, [M+H]⁺475.2135, found 475.2078.

### (1-(9-ethyl-9H-carbazol-3-yl)-5-phenyl-1H-1,2,3-triazol-4-yl) (4-nitrophenyl) methanol (4f)

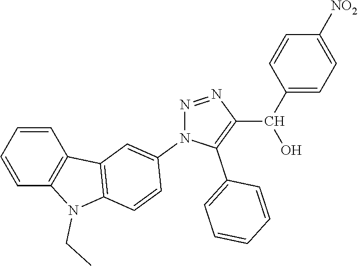 [SMILES: CCn1c2ccccc2c2cc(-n3nnc(C(O)c4ccc([N+](=O)[O-])cc4)c3-c3ccccc3)ccc21]

Bone white solid (0.15 mmol, 19%, 74.1 mg). R_{f}=0.54 (Hexane/Ethyl Acetate, 1:1); 1H-NMR (CDCl₃, 500 MHz) δ 1.44 (t, J=7.2 Hz, 3H), 3.59 (d, J=6.2 Hz, 1H), 4.36 (q, J=7.2 Hz, 2H), 6.13 (d, J=5.9 Hz, 1H), 7.19-7.15 (m, 2H), 7.25 (t, J=7.5 Hz, 1H), 7.37-7.27 (m, 6H), 7.44 (d, J=8.2 Hz, 1H), 7.52 (t, J=7.7 Hz, 1H), 7.61 (d, J=8.7 Hz, 1H), 7.95 (d, J=7.8 Hz, 1H), 8.05 (d, J=1.8 Hz, 1H), 8.15 (d, J=8.8 Hz, 2H); ¹³C (CDCl₃, 100 MHZ) δ 13.78, 37.79, 68.02, 108.59, 108.92, 117.56, 119.56, 120.68, 122.36, 122.58, 123.02, 123.49, 126.42, 126.68, 127.41, 127.79, 128.82, 129.48, 129.86, 135.50, 139.65, 140.62, 146.07, 147.28, 149.12; FTMS (ESI) m/z calculated for C₂₉H₂₃N₅O₃, [M+H]⁺490.1880, found 490.1827. (1-(9-ethyl-9H-carbazol-3-yl)-5-phenyl-1H-1,2,3-triazol-4-yl) (p-tolyl) methanol (4g)

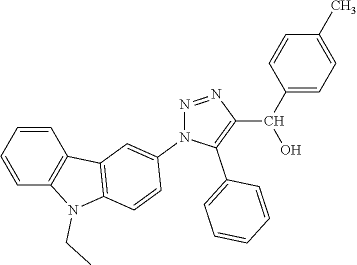 [SMILES: CCn1c2ccccc2c2cc(-n3nnc(C(O)c4ccc(C)cc4)c3-c3ccccc3)ccc21]

Light brown solid (0.08 mmol, 10%, 36.15 mg). R_{f}=0.2 (Hexane/Ethyl Acetate, 3:1); 1H-NMR (CDCl₃, 500 MHz) δ 1.46 (t, J=7.2, 3H), 2.07 (s, 1H), 2.37 (s, 3H), 4.38 (q, J=7.2 Hz, 2H), 5.92 (s, 1H), 7.17 (dd, J=7.0, 5.4 Hz, 4H), 7.37-7.23 (m, 7H), 7.41 (d. J=8.0 Hz, 2H), 7.45 (d, J=8.2 Hz, 1H), 7.52 (t, J=7.6 Hz, 1H), 7.98 (d, J=7.8 Hz, 1H), 8.08 (d, J=1.7 Hz, 1H); ¹³C NMR (100 MHZ, CDCl3) δ13.80, 21.15, 37.81, 68.69, 108.56, 108.90, 117.66, 119.51, 120.74, 122.49, 122.79, 123.03, 126.60, 126.84, 126.92, 128.19, 128.66, 129.09, 129.19, 129.95, 134.70, 137.50, 139.56, 139.61, 140.63, 147.06; FTMS (ESI) m/z calculated for C₃₀H₂₆N₄O₂, [M+H]⁺459.2186, found 459.2130.

### (1-(9-ethyl-9H-carbazol-3-yl)-5-phenyl-1H-1,2,3-triazol-4-yl) (phenyl) methanol (4h)

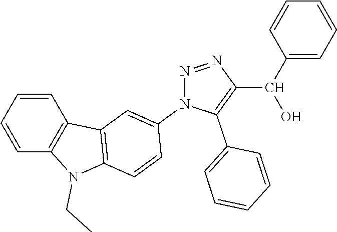 [SMILES: CCn1c2ccccc2c2cc(-n3nnc(C(O)c4ccccc4)c3-c3ccccc3)ccc21]

Cream solid (0.20 mmol, 29%, 90.6 mg). R_{f}=0.57 (Hexane/Ethyl Acetate, 1:1); 1H-NMR (CDCl₃, 500 MHz) δ 1.45 (t, J=7.2 Hz, 3H), 3.34 (d, J=5.0 Hz, 1H), 4.37 (q, J=7.2 Hz, 2H), 5.99 (s, 1H), 7.17 (dd, J=1.5, 8.4 Hz, 2H), 7.25 (s, 1H), 7.28-7.30 (m, 3H), 7.31-7.33 (m, 2H), 7.35 (dd, J=2.2, 5.7 Hz, 2H), 7.37 (d, J=7.2 Hz, 1H), 7.45 (d, J=8.2 Hz, 1H), 7.52 (d, J=7.4 Hz, 1H), 7.53 (dd, J=1.2, 7.0 Hz, 1H), 7.54 (d, J=1.0 Hz, 1H), 7.98 (d, J=7.7 Hz, 1H), 8.10 (d, J=1.3 Hz, 1H) ppm; ¹³C (CDCl₃, 100 MHz) δ 13.77, 37.77, 68.80, 108.52, 108.86, 117.61, 119.47, 120.70, 122.43, 122.73, 122.98, 126.56, 126.81, 126.82, 127.71, 128.10, 128.42, 128.63, 129.10, 129.92, 134.85, 139.57, 140.59, 142.35, 146.93 ppm; FTMS (ESI) m/z calculated for C₂₉H₂₄N₄O, [M+H]⁺445.2023, found 445.2008.

### (1-(9-Ethyl-9H-carbazol-3-yl)-5-phenyl-1H-[1,2,3]triazol-4-yl)-thiophen-2-yl-methanone (10)

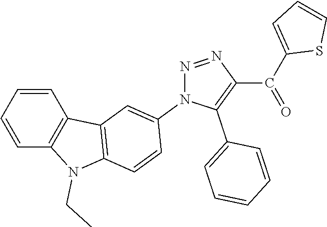 [SMILES: CCn1c2ccccc2c2cc(-n3nnc(C(=O)c4cccs4)c3-c3ccccc3)ccc21]

In a 50 mL three-neck round-bottom flask containing phenylacetylene 121 μL (1.1 mmol) under N₂ atmosphere, a solution of (phenylethynyl) magnesium bromide 1.4 mL (1.4 mmol) in THF was added dropwise at room temperature. After the Grignard reagent was added, the reaction mixture was placed in an oil bath and heated at 50° C. for 15 minutes. The mixture was cooled at room temperature and neat azide (3-azido-9-ethyl-9H-carbazole) 0.1890 g (0.8 mmol) was added. The reaction mixture was heated to 50° C. for 1 hour, cooled at room temperature and 2-Thiophenercarbonyl chloride 118 μL (1.1 mmol) was added to the mixture at room temperature. After 2.5 hour the reaction was analyzed by TLC and 2-Thiophenercarbonyl chloride 53 μL (0.5 mmol) was added to the mixture. After 1 hour the reaction was completed (analyzed by TLC) and the quenching with 10% Ammonium chloride was performed. The products were extracted with ethyl acetate (3×) and the organic phase was washed with brine, dried, filtered and concentrated under reduced pressure. The crude was purified with Hexane/Ethyl Acetate gradient to obtain 20.8 mg (0.05 mmol, 6%) of a yellow solid. Purity was verified by TLC, NMR spectroscopy and HPLC-MS: R_{f}=0.55 (Hexane/Ethyl Acetate, 3:1);

¹H-NMR (CDCl₃, 500 MHz) δ 1.45 (t, J=7.0 Hz, 3H), 4.37 (q, J=7.0, 2H), 7.23-7.54 (m, 11H), 7.74 (d, J=4.0, 1H), 7.99 (d, J=8.0 Hz, 1H), 8.09 (d, J=1.5 Hz, 1H), 8.65 (d, J=3.0, 1H); ¹³C (CDCl₃, 100 MHz) δ 13.79, 37.83, 108.65, 108.96, 117.83, 119.64, 120.74, 122.39, 122.77, 123.08, 126.28, 126.74, 127.44, 128.24, 128.30, 129.68, 130.46, 134.82, 136.12, 139.75, 140.65, 141.27, 142.79, 143.42, 177.97; FTMS (ESI) m/z calculated for C₂₇H₂₀N₄OS, [M+H]⁺449.1437, found 449.1384.

### Procedure for 4-Cyano Derivative

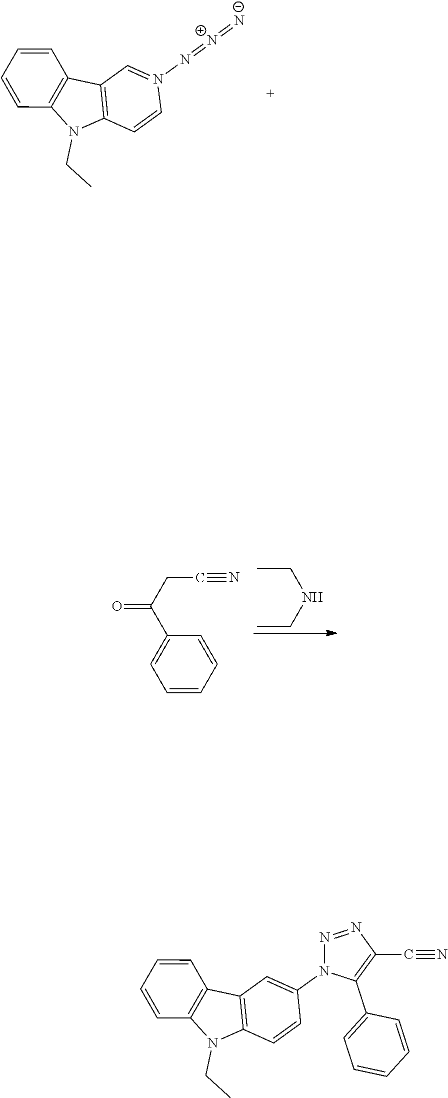

### 1-(9-ethyl-9H-carbazol-3-yl)-5-phenyl-1H-1,2,3-triazole-4-carbonitrile (5)

In a 10 mL round-bottom flask were added 472 mg (2.0 mmol) of compound 3-azido-9-ethyl-9H-carbazole), 290 mg (2.0 mmol) of benzalacetonitrile and 41 μL diethyl amine (0.4 mmol) in 1.5 mL DMSO. After heating for 3 hours at 70° C., the reaction mixture was extracted with ethyl acetate and water and purified via silica gel flash chromatography with hexanes/ethyl acetate (5:1 to 1:1) as eluent. Product 1-(9-ethyl-9H-carbazol-3-yl)-5-phenyl-1H-1,2,3-triazole-4-carbonitrile was obtained as a cream-colored solid.

¹H-NMR (CDCl₃, 500 MHz) δ 1.45 (t, J=7.3 Hz, 3H), 4.43 (q, J=8.3 Hz, 2H), 7.31 (t, J=7.2, 1H), 7.34 (dd, J=9.0, 2.0 Hz, 1H), 7.39-7.51 (m, 7H), 7.57 (t, J=7.7, Hz, 1H), 8.03 (d, J=7.8 Hz, 1H), 8.14 (d, J=2.0 Hz, 1H) ppm; ¹³C (CDCl₃, 100 MHz) δ 13.84, 37.94, 109.03, 109.13, 112.45, 117.81, 119.89, 120.24, 120.84, 122.27, 122.58, 123.35, 123.75, 126.82, 127.06, 128.99, 129.31, 130.84, 140.11, 140.78, 143.24 ppm.

### Procedure for 4-Carboxyl Ester and Derived Products

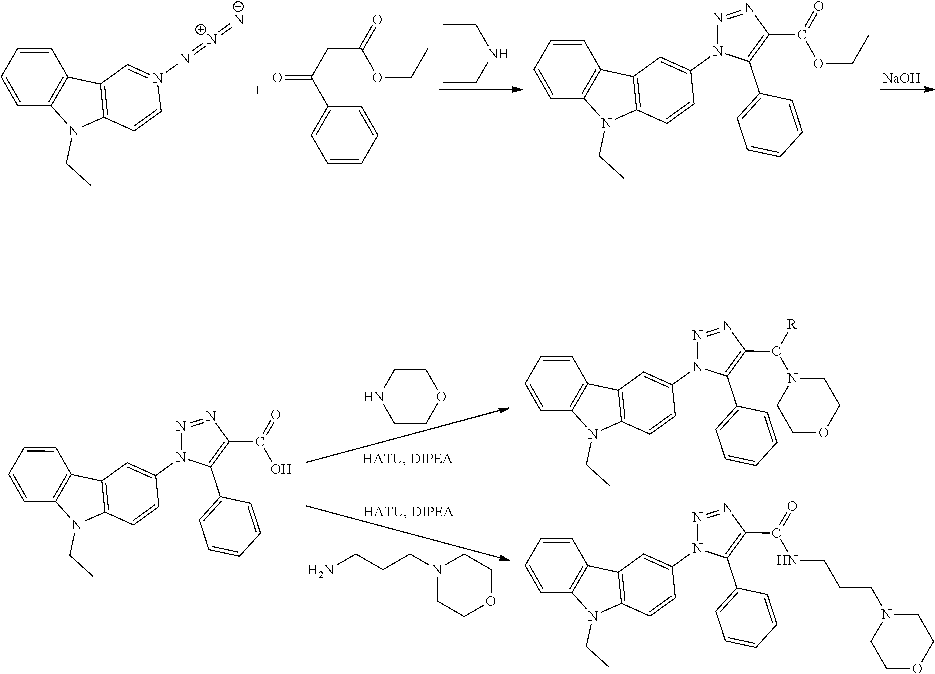

### Ethyl 1-(9-ethyl-9H-carbazol-3-yl)-5-phenyl-1H-1,2,3-triazole-4-carboxylate (6)

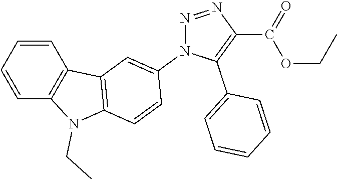 [SMILES: CCOC(=O)c1nnn(-c2ccc3c(c2)c2ccccc2n3CC)c1-c1ccccc1]

In a 10 mL flask was added 4/2 mg (2.0 mmol) ₃-azido-9-ethyl-9H-carbazole), 384 mg (2.0 mmol) ethyl benzoyl acetate, and 10 μL diethyl amine (0.4 mmol) in 1.5 mL DMSO. After stirring overnight at 70° C., the reaction mixture was worked up via extraction with ethyl acetate and water. Purification via silica gel flash chromatography provided 261 mg (0.6 mmol) of product ethyl 1-(9-ethyl-9H-carbazol-3-yl)-5-phenyl-1H-1,2,3-triazole-4-carboxylate as an off-white solid.

¹H-NMR (CDCl₃, 500 MHz) δ 1.38 (t, J=8.4 Hz, 3H), 1.46 (t, J=7.2 Hz, 3H), 4.38 (q, J=7.3 Hz, 2H), 4.42 (q, J=7.1, 2H), 7.25-7.30 (m, 3H), 7.33-7.40 (m, 6H), 7.46 (d, J=8.2 Hz, 1H), 7.54 (dd, J=7.7, 1.2 Hz, 1H), 8.00 (d, J=7.7 Hz, 1H), 8.07 (d, J=2.0 Hz, 1H) ppm; ¹³C (CDCl₃, 100 MHz) δ 13.80, 14.22, 37.84, 61.16, 108.62, 108.97, 117.86, 119.64, 120.77, 122.40, 122.80, 123.06, 126.16, 126.74, 124.47, 128.26, 129.71, 130.42, 136.76, 139.75, 140.67, 141.16, 161.24 ppm.

### 1-(9-Ethyl-9H-carbazol-3-yl)-5-phenyl-1H-1,2,3-triazole-4-carboxylic acid (7)

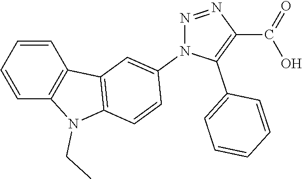 [SMILES: CCn1c2ccccc2c2cc(-n3nnc(C(=O)O)c3-c3ccccc3)ccc21]

In a 20 mL vial, 205 mg (0.5 mmol) compound 6 was dissolved in 4.5 mL dichloromethane and 0.5 mL 3M sodium hydroxide in methanol was added. After stirring for 3 hours at room temperature, water was added, and the aqueous phase extracted 2× with ethyl acetate. The aqueous phase was acidified with 1.0 N HCl until a pH of 2 was obtained, and subsequently extracted with ethyl acetate. After drying the organic phase with sodium sulfate, the solvents were evaporated to obtain 160 mg (0.42 mmol) ₁-(9-Ethyl-9H-carbazol-3-yl)-5-phenyl-1H-1,2,3-triazole-4-carboxylic acid of a white solid.

¹H-NMR (CDCl₃, 500 MHZ) δ 1.47 (t, J=7.3 Hz, 3H), 4.40 (q, J=7.3 Hz, 2H), 7.26-7.31 (m, 3H), 7.33-7.42 (m, 6H), 7.47 (d, J=8.2 Hz, 1H), 7.55 (t, J=7.7, 1H), 8.02 (d, J=7.8 Hz, 1H), 8.09 (d, J=2.0 Hz, 1H) ppm; ¹³C (CDCl₃, 100 MHz) δ 13.81, 37.87. 108.71, 109.02, 117.88, 119.72, 120.78, 122.36, 122.73, 123.12, 125.35, 126.83, 127.28, 128.41, 130.03, 130.42, 139.84, 140.67, 141.66, 163.17 ppm

### (1-(9-ethyl-9H-carbazol-3-yl)-5-phenyl-1H-1,2,3-triazol-4-yl) (morpholino) methanone (8)

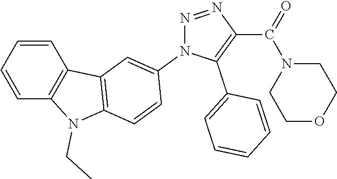 [SMILES: CCn1c2ccccc2c2cc(-n3nnc(C(=O)N4CCOCC4)c3-c3ccccc3)ccc21]

In a 20 mL vial, to 1.5 mL DMF was added 48 mg (0.125 mmol) 7, 13 μL (0.15 mmol) morpholine, 56 mg (0.15 mmol) HBTU, and 26 μL (0.15 mmol) DIPEA. The solution was stirred overnight at room temperature, extracted with ethyl acetate/water and purified via silica gel flash chromatography (hexanes/ethyl acetate 8:1 to 1:1) to obtain (1-(9-ethyl-9H-carbazol-3-yl)-5-phenyl-1H-1,2,3-triazol-4-yl) (morpholino) methanone as an off-white solid.

¹H-NMR (CDCl₃, 500 MHz) δ 1.48 (t, J=7.2 Hz, 3H), 3.65 (bs, 2H), 3.78 (bs, 2H), 3.82 (m, 4H), 4.40 (q, J=7.2 Hz, 2H), 7.26-7.40 (m, 9H), 7.47 (d, J=8.2 Hz, 1H), 7.55 (t, J=7.3 Hz, 1H), 8.01 (d, J=7.8 Hz, 1H), 8.11 (d, J=2.0 Hz, 1H) ppm; ¹³C (CDCl₃, 100 MHz) δ 13.82, 37.86, 42.76, 47.83, 66.78, 67.03, 108.70, 108.99, 117.79, 119.65, 120.76, 122.41, 122.80, 123.14, 126.21, 126.76, 127.67, 128.65, 129.58, 129.89, 138.41, 139.66, 139.78, 140.69, 161.54 ppm.

### 1-(9-ethyl-9H-carbazol-3-yl)-N-(3-morpholinopropyl)-5-phenyl-1H-1,2,3-triazole-4-carboxamide (9)

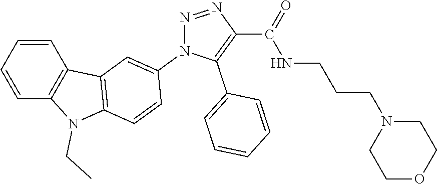 [SMILES: CCn1c2ccccc2c2cc(-n3nnc(C(=O)NCCCN4CCOCC4)c3-c3ccccc3)ccc21]

In a 20 mL vial, to 1.5 mL DMF was added 48 mg (0.125 mmol) 7, 22 μL (0.15 mmol) 4-(3-aminopropyl) morpholine, 56 mg (0.15 mmol) HBTU, and 26 μL (0.15 mmol) DIPEA. The solution was stirred overnight at room temperature, extracted with ethyl acetate/water and purified via silica gel flash chromatography (methanol/DCM 2% to 5%) to obtain 1-(9-ethyl-9H-carbazol-3-yl)-N-(3-morpholinopropyl)-5-phenyl-1H-1,2,3-triazole-4-carboxamide as an off-white solid.

¹H-NMR (CDCl₃, 500 MHz) δ 1.45 (t, J=7.2 Hz, 3H), 1.87 (q, J=6.0 Hz, 2H), 2.59, (bm, 6H), 3.59 (m, 2H), 3.88 (bs, 4H), 4.38 (q, J=7.2 Hz, 2H), 7.24-7.38 (m, 6H), 7.38-7.48 (m, 3H), 7.53 (t, J=7.6 Hz, 1H), 8.00 (d, J=7.8 Hz, 1H), 8.08 (bs, 1H), 8.42 (bs, 1H), ppm; ¹³C (CDCl₃, 100 MHz) δ 13.80, 25.30, 37.83, 38.57, 53.79, 57.69, 66.71, 108.62, 108.97, 117.82, 119.62, 120.77, 122.42, 122.83, 123.08, 126.19, 126.71, 127.73, 128.12, 129.45, 130.69, 132.09, 138.78, 139.04, 139.71, 140.67, 160.65 ppm.

### BIOLOGICAL ASSAYS

### Cell Culture

MDA-MB-231, MDA-MB-468, MCF7, MCF10A, HMEC, and A549 cells were purchased from ATCC. MDA-MB-435 cell line used is a metastatic variant from a HER2++ breast cancer (HER2-BM) and is a kind gift from Dr. Danny Welch (University of Kansas Cancer Center). Cells were cultured and maintained as previously described.

### Initial Screening

For initial screening, the GIso of compounds 4a-4h in the cell lines MDA-MB-231, MDA-MB-435 and MCF-7 was determined via the SRB-assay for 48 hrs as follows:

A stock solution of compounds was prepared at 50 mM in 100% DMSO. For cells preparation, a flask of 75 cm² or 25 cm² were used for 2.6×10⁵ cells/mL or 1.44×10⁵ cells/mL, respectively, with an 80-90% of confluence. Cells were washed with PBS and trypsinized. The concentration of cells was determined using a 1:2 dilutions with Trypan Blue and a hemocytometer. After cell count, the concentration was adjusted to have a 7.0-10.0×10⁴ cells/mL. Approximately 100 μL of cells suspension, compounds, control positive and control negative were added in triplicates to a 96 well plate. Positive control used was doxorubicin, and the negative control was DMSO 0.1%. All compounds (4a-h) at 50, 25, 12.5, 6.3, and 1.6 μM were incubated with cells at 37° C. for 48 hrs. For fixation, cold TCA 50% was used and incubated at 4° C. for 1 h. Wells were washed and dried prior to tincture with 100 μL of SRB 0.4%. To remove excess of SRB, acetic acid was used. For analysis, TRIS-BASE Solution (pH=10.5) was used and shaken prior to reading using an ELISA reader at 540 nm and the software SoftMax Pro 4.8. For each compound, 50% growth inhibition (GI₅₀) was calculated from Sigmoidal dose-response curves (variable-slope) that were generated with data obtained from experiments carried out in triplicates (GI₅₀ values were generated with GraphPad Prism V. 6.02, GraphPad Software, Inc.).

In addition, the inhibition of migration of MDA-MB-231 was determined via the following procedure:

Prior to assays, cells were grown until 80-90% confluence was observed. We used a 75 cm² flask for 2.6×10⁵ cells/mL in 10 mL and for a 25 cm² flask 1.44×10⁵ cells/mL in 5 mL. The cells were washed with PBS to remove all traces of FBS. We added 2 mL for a 25 cm² flasks or 4 mL for a 75 cm² flasks trypsin, and incubated 5-10 min at 37° C. At the end of the incubation time, cells were re-suspended and counted with hemocytometer using 1:2 dilutions with Trypan blue. Subsequently, cell viability was calculated. In a 12 well tissue culture plate, we used the plastic pattern to draw a fine space with a fine marker on the bottom of the plate. Cells were seeded at 1.5-2.2×10⁵ cells/mL in 1 mL and incubated for 24 h. Cells were then rinsed with PBS and incubated in starving media (0.5% FBS) overnight. All controls and drugs were tested in triplicate. The negative control for each drug was prepared according to the drug's DMSO concentration. Drugs were diluted and the final concentration at each well was GI₅₀/5 on MDA-MB-231 cells (or at concentrations that do not affect cell viability). The wound was made using a sterile pipette tip of 200 μL. Cells were then rinsed very gently with media without FBS and media with negative controls was added. After a 24 h incubation, the gap distance was evaluated using the software Lumera Infinity Analyze 6.4.0. Pictures were taken at 0, 8, 12 and 24 h using a 10× objective in an Inverted Laboratory Microscope Leica DM IL LED, and an Infinity1-3 3.1 Megapixel USB 2.0 camera CMOS. The percentage of migration was calculated using the following formula:

$$100 - [ ( {X}_{0} / {\ddot{X}}_{0} ) ] * 100 for time 0 h measurements$$

$$100 - [ ( {X}_{24} / {\ddot{X}}_{0} ) ] * 100 for time 24 h measurements$$

The results of these initial screenings are compiled in Table 1.

TABLE 1
Structure and initial screening of biological activities

| | | MDA-MB- | MDA- | MCF-7 | Inh. of |
|---|---|---|---|---|---|
| | | 231 GI₅₀ | MB435 | GI₅₀ | migration |
| # | Structure | (μM) | GI₅₀ (μM) | (μM) | (%) |
| 4a | 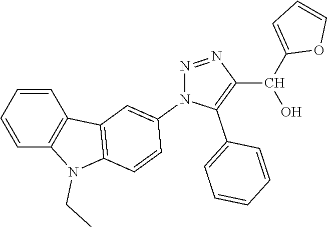 [SMILES: CCn1c2ccccc2c2cc(-n3nnc(C(O)c4ccco4)c3-c3ccccc3)ccc21] | 17.5 | 0.36 | 20.6 | 55 ± 13.58 (at 6.6 μM) |
| 4b | 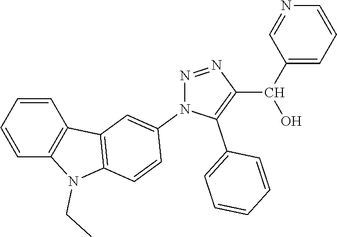 [SMILES: CCn1c2ccccc2c2cc(-n3nnc(C(O)c4cccnc4)c3-c3ccccc3)ccc21] | 8.0 | 0.08 | 0.04 | 61.4 ± 9.08 (at 1.6 uM) 25.2 ± 10.06 (at 0.5 μM) |
| 4c | 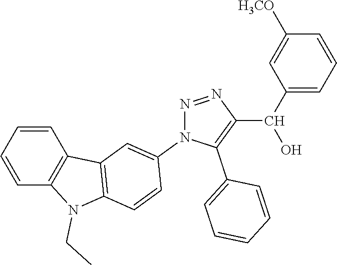 [SMILES: CCn1c2ccccc2c2cc(-n3nnc(C(O)c4cccc(OC)c4)c3-c3ccccc3)ccc21] | 17.2 | 1.6 | 3.2 | 50.8 ± 11.42 (at 10 μM) |
| 4d | 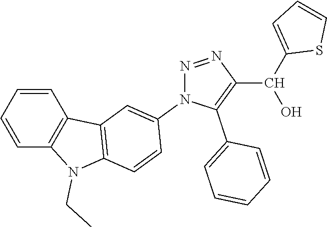 [SMILES: CCn1c2ccccc2c2cc(-n3nnc(C(O)c4cccs4)c3-c3ccccc3)ccc21] | 20.4 | 19.6 | 12.0 | 57.5 ± 10.58 (at 4.1 μM) 8.7 ± 5.73 (at 0.5 μM) |
| 4e | 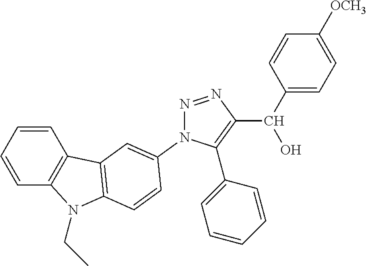 [SMILES: CCn1c2ccccc2c2cc(-n3nnc(C(O)c4ccc(OC)cc4)c3-c3ccccc3)ccc21] | 26.2 | 32.9 | 10.6 | 57.0 ± 12.26 (at 5.3 μM) 36.1 ± 15.09 (at 0.5 μM) |
| 4f | 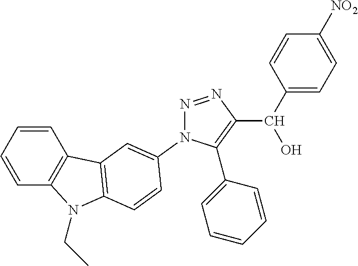 [SMILES: CCn1c2ccccc2c2cc(-n3nnc(C(O)c4ccc([N+](=O)[O-])cc4)c3-c3ccccc3)ccc21] | 11.2 | 7.5 | 9.7 | 61.1 ± 6.12 (at 2.2 μM) 22.1 ± 6.88 (at 0.5 μM) |
| 4g | 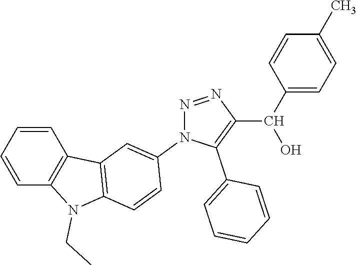 [SMILES: CCn1c2ccccc2c2cc(-n3nnc(C(O)c4ccc(C)cc4)c3-c3ccccc3)ccc21] | 7.9 | 2.2 | 13.5 | 15.1 ± 10.67 (at 1.6 μM) |
| 4h | 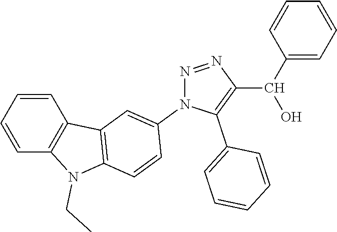 [SMILES: CCn1c2ccccc2c2cc(-n3nnc(C(O)c4ccccc4)c3-c3ccccc3)ccc21] | 24.3 | 22.6 | 9.6 | 65.4 ± 11.89 (at 4.9 μM) 11.1 ± 10.13 (at 0.5 μM) |
| 10 | 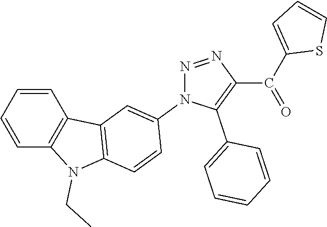 [SMILES: CCn1c2ccccc2c2cc(-n3nnc(C(=O)c4cccs4)c3-c3ccccc3)ccc21] | 0.915 | 23.27 | 0.19 | |

TABLE 2
Cytotoxicity determination of compounds 5-9 at a fixed concentration of 500 nM in
MDA-MB-231 tumor cells.

| | | % growth | | |
|---|---|---|---|---|
| | | inhibition | GI50 | |
| | | MDA-MB- | MDA-MB- | % Rac |
| Comp # | Structure | 231ª | 435^{b} | inhibitionc |
| MBQ- 167 | 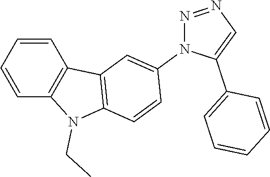 [SMILES: CCn1c2ccccc2c2cc(-n3nncc3-c3ccccc3)ccc21] | 36% | 145 nM | 250 nM: ~60% |
| 5 | 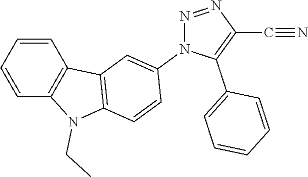 [SMILES: CCn1c2ccccc2c2cc(-n3nnc(C#N)c3-c3ccccc3)ccc21] | 46% | 45 nM | 250 nM: >90% 100 nM: >90% 50 nM: >90% 10 nM: ~30% |
| 6 | 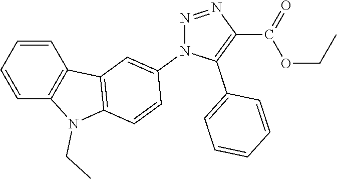 [SMILES: CCOC(=O)c1nnn(-c2ccc3c(c2)c2ccccc2n3CC)c1-c1ccccc1] | 32% | ~5 UM | 250 nM: ~60- 70% |
| 7 | 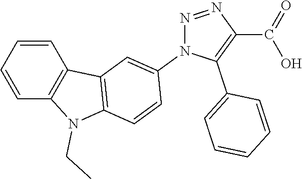 [SMILES: CCn1c2ccccc2c2cc(-n3nnc(C(=O)O)c3-c3ccccc3)ccc21] | 29% | — | — |
| 8 | 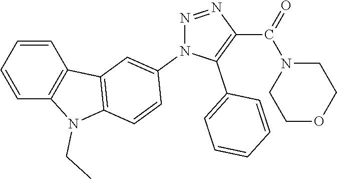 [SMILES: CCn1c2ccccc2c2cc(-n3nnc(C(=O)N4CCOCC4)c3-c3ccccc3)ccc21] | -11% | ~1 uM | 250 nM : <20% |
| 9 | 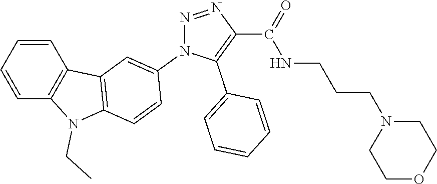 [SMILES: CCn1c2ccccc2c2cc(-n3nnc(C(=O)NCCCN4CCOCC4)c3-c3ccccc3)ccc21] | 13% | >10 uM | 250 nM : <20% |

^{a}Cytotoxicity determination of compounds 5-9 at a fixed concentration of 250 nM for 48 hours in MDA-MB-231 tumor cells.
^{b}Growth inhibition compared with MBQ-167 curves at concentrations ranging from 0-1000 nM for 72 hrs, using the MTT assay.
^{c}Determination of Rac inhibitory activity at 250 nM via pulldowns using a GST-fusion protein of the Cdc42-rac interactive binding (CRIB) domain of P21-activated kinase (PAK).

### Cell Viability Assay

Equal number of cells were seeded in a 24 well plate and incubated with vehicle or compounds according to this disclosure at concentrations between 1 and 0.001 nM for 72 and 120 hrs. Using CellTiter 96 Non-Radioactive Cell Proliferation Assay (Promega, Corp.) according to manufactures' instructions, formazan was used as an indicator of viable cells at an absorbance 570 nm in a Tecan Microplate Reader M100. Data was calculated as fold change from vehicle and fitted using the 4′parameter logic nonlinear regression model in GraphPad Prism software. (n=3 biologic replicates: with 3 technical replicates each).

### Apoptosis Assay

Equal number of cells were seeded incubated with vehicle or compounds according to this disclosure at 250 or 500 nM for 48 hrs. Apoptosis was measured using a Caspase-Glo3/7 Luminescence Assay Kit as per manufacturer's instructions (Promega, Corp.). Briefly, Caspase-3/7 Glo reagent was added and incubated at room temperature for 60 min and Caspase-3/7 activities were determined by detecting luminescence using Tecan Microplate Reader M100. (n=3 biologic replicates: with 3 technical replicates each).

### Wound Healing Assay

MDA-MB-231 cells plated on 12-well plates at equal cell density until confluent. Then the media was changed to 1% FBS, and a single scratch was made in the center of the monolayer culture with a pipet tip. Compounds were added immediately at 250, or 500 nmol/L and images were digitally acquired from a Keyence microscope (10×) at 0, 12, and 24 hours. The scratch area was quantified in ImageJ using the Wound Healing Tool (n=3 biologic replicates: with 3 technical replicates each).

### Actin Cytoskeleton Staining

Cells were grown on glass coverslips and treated with vehicle or 4c for 24h. Lung cancer cells that were stimulated with epidermal growth factor (EGF) were serum starved prior to stimulation with 200 ng/ml EGF for 5 min. Following fixation in 4% formaldehyde, cells were stained for F-actin with Rhodamine phalloidin and visualized by fluorescence microscopy. 5 random fields were scored for the number of ruffles.

### Rac Activation Assay

MDA-MB-231 triple negative breast cancer cells were seeded at equal density and treated with vehicle or Compound 4c for 24 hrs at various concentrations. Rac-GTP was detected using Rac Activity Assay, as per manufacturer's instructions (Cell Signaling). Briefly, total cell lysates were quantified and added equally to Pak-PBD beads. After 1 hr of incubation, beads were washed and Rac1-GTP (in pellets) or total Rac1 (in cell lysates) was detected by western blot using Rac1 antibody. (n=3 biological replicates).

### Rac1 GEF Assay

MDA-MB-231 cells in culture medium were lysed in 1% Triton X-100, 20 mM HEPES, pH 7.4, 150 mM NaCl, 5 mm MgCl₂, and protease inhibitors and processed. Equal amounts of protein from cleared lysates were incubated for 1 h at 4° C. with glutathione-agarose beads conjugated to GST-Rac1(G15A) nucleotide-free mutant (Cell Biolabs, San Diego. CA) that were preincubated (for 1 h) with vehicle or 8 μm EHop-016 or 0.25 μM Compound 4c. The beads were washed, and the lysates and pull-downs were immunoblotted for Tiam-1, Trio, Ect2, p-REX, or Vav2.

### Nucleotide Association Assay

Purified proteins Rac1, Cdc42 were purchased from Cytoskeleton. For the assay, 2 μM of Rac1-GDP was added to 384 well plates containing 3 μM BODIPY-FL-GTPγS, 3 or 10 μM of MBQ-167 or compounds according to the present disclosure in 20 mM Tris HCl pH 7.5, 100 mM NaCl, and 1 mM EDTA.

Recombinant Protein Nucleotide Loading—To load GST-Sepharose-bound small GTPases, the bead-bound GTPases were incubated in exchange buffer (20 mM TrisHCl, 50 mM NaCl, 500 μM fluorescent N-methylanthraniloyl (mant) deriv-atives of guanine nucleotides (mant-GDP and mant-GMPPNP, Roche Applied Science), 20 mM (NH4)₂SO₄), for 1 min at 37° C., before three washes in cold equilibration buffer (20 mM TrisHCl, 50 mM NaCl, 1 mM MgCl2). Nucleotide-loaded protein was then eluted from the beads using elution buffer (20 mM TrisHCl, 50 mM NaCl, 1 mM MgCl₂, 0.1 mM glutathione) for 10 min on ice. To load His6-tagged proteins, the relevant protein was incubated in exchange buffer for 1 min at 37° C., before being separated from excess nucleotide in a Zeba Desalting Spin Column (Pierce Biotechnology), prepared as per manufacturer's protocol.

### Results

### Inhibition of Cell Viability and Apoptosis in Metastatic Cancer Cells

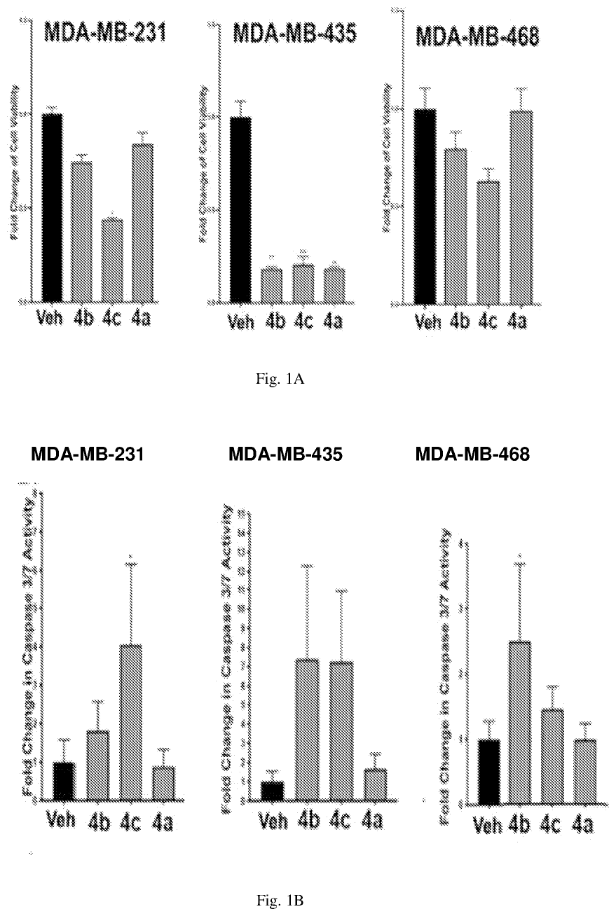

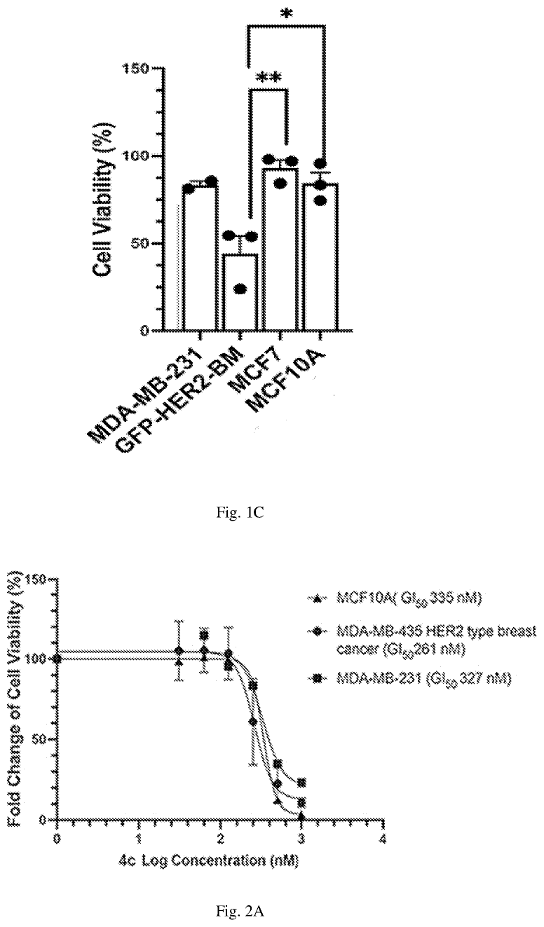

Cell viability was screened with 500 nM of compounds according to the present disclosurein MDA-MB-231 (triple negative breast cancer (TNBC), MDA-MB-435 (GFP-HER2-++), and MDA-MB-468 (TNBC) breast cancer cell lines. Compound 4c significantly inhibited MDA-MB-231 cell viability by <50% at 500 nM. Compounds 4a, 4b, and 4c demonstrated significant ˜80% decreases in cell viability at 500 nM in the human epidermal growth factor2 (HER2) positive MDA-MB-435 cell line. Decreased cell viability in response to 4c was a non-statistically significant trend in the more aggressive epidermal growth factor receptor (EGFR)++ TNBC cell line MDA-MB-468 (FIG. 1A).

Next, the effect of the compounds according to this disclosure on apoptosis via analysis of caspase 3/7 activity in the breast cancer cell lines was tested. The compounds according to the present disclosure inhibited cell viability: 4c in the MDA-MB-231 cell line, 4b and 4c in the MDA-MB-435 cell line, and 4b in the MDA-MB-468 cell line. Compound 4b also significantly increased Caspase 3/7 the activity in the MDA-MB-468 cell line, and thus, apoptosis. Compound 4c induced the activity of Caspase 3/7 three to five times more compared to the vehicle in the MDA-MB-231 and MDA-MB-435 breast cancer cell lines. Compound 4b also increased apoptosis in all three cell lines (FIG. 1B). 4c was not effective in the MDA-MB-231, MCF7, or MCF-10 cell lines at 250 nM, but inhibited MDA-MB-435 cells by ˜60% at 250 nM, demonstrating its superior efficacy in this HER2 positive metastatic cancer cell line (FIG. 1C). Therefore, 4c was selected for further analysis as an anti-cancer compound in TNBC cells.

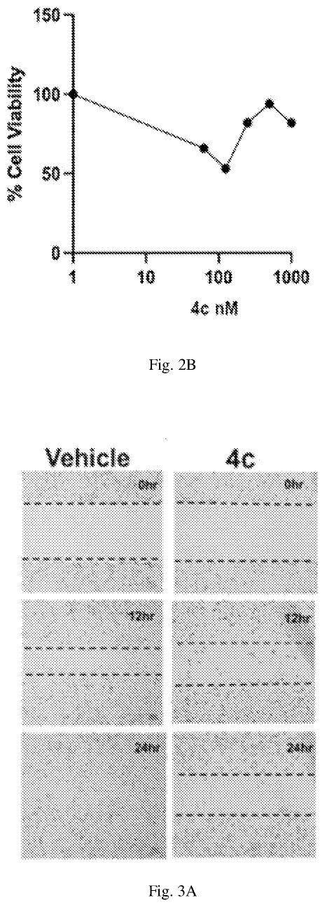

The concentration dependence of the selected compound 4c was analyzed to determine the half maximal growth inhibitory concentration (GI₅₀). 4c demonstrated GIsos in the nanomolar range of 261 nM in the MDA-MB-435 cell line and 327 nM in the MDA-MB-231 cells and a GIso of 335 nM in the transformed mammary epithelial MCF10A cell line (FIG. 2A). However, 4c did not affect non-transformed human mammary epithelial cells (HMEC), indicating that 4c is not toxic to non-cancer mammary epithelial cells (FIG. 2B). The GI₅₀ for breast cancer cell viability inhibition of ˜0.3 mM for 4c is comparable with the GIso for MBQ-167.

### Compound 4c and Inhibition of Breast Cancer Cell Migration and Actin Cytoskeletal Extensions

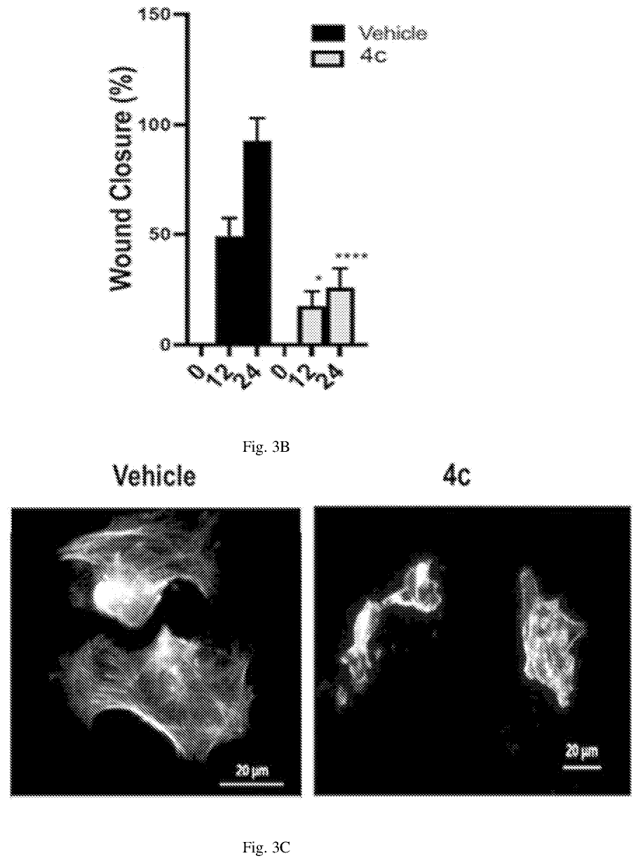

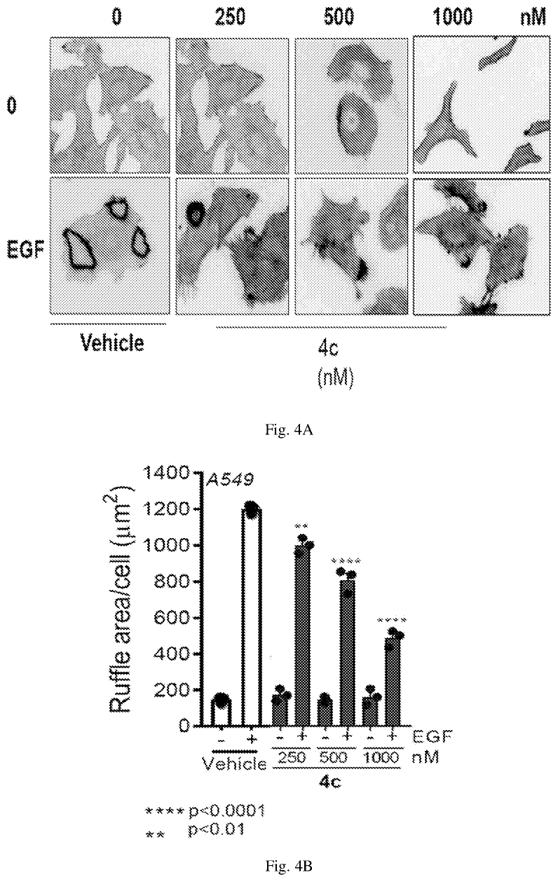

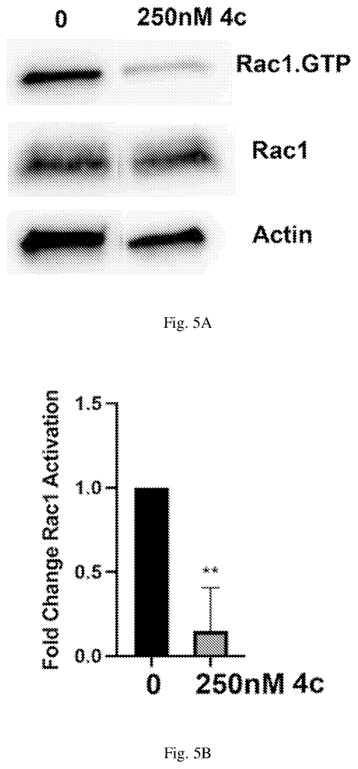

To test the anti-migratory effect of 4c, we conducted wound healing assays using confluent MDA-MB-231 cells. After generation of the wound, 250 nM of 4c were added and the area of the wound quantified from digital images at 0, 12 and 24 hr. Results show that the wound area was maintained at 12 and 24 hr for cells treated with 4c, in a statistically significant manner compared to vehicle (FIGS. 3A, 3B). To determine the effect on the actin cytoskeleton, MDA-MB-231 cells were treated with vehicle or 4c for 12 hr at 250 nM, and the actin cytoskeleton was stained with Rhodamine phallodin to identify changes in F-actin, which are required for cell migration. FIG. 3C shows the cell phenotype, where vehicle cells are spread with an actin cytoskeleton organized into stress fibers and membrane ruffles. However, 4c treated cells were smaller and less spread. Without being bound by any theory, 4c may cause changes in cell morphology and loss of actin filaments, thus impeding cell migration.

The effect of 4c was further tested in the A549 lung adenocarcinoma cell line. Serum starved lung cancer cells were incubated with 4c for 24 h, then stimulated with epidermal growth factor (EGF) to induce actin-based membrane ruffles. Results show that treatment with 4c reduced membrane ruffles in response to EGF by 20% at 250 nM, ˜38% at 500 nM, and 58% at 1000 nM in a statistically significant manner (FIGS. 4A, 4B).

Therefore, without being bound by any theory, compound 4c may serve as a promising anticancer agent in breast and lung cancers.

### Compound 4c and Inhibition of Rac Activation by Inhibiting the Interaction of the Guanine Nucleotide Exchange Factor (GEF) Vav2 with Rac1

To determine the potential of Compound 4c to inhibit Rac1, which directs cell migration and lamellipodia formation, a pulldown assay was performed using the Rac/Cdc42 interacting domain of their downstream effector p21-activated kinase (PAK), which only binds GTP bound active Rac. FIGS. 5A and 5B show that Compound 4c at 250 nM reduces Rac1 activation by GTP loading by 85%.

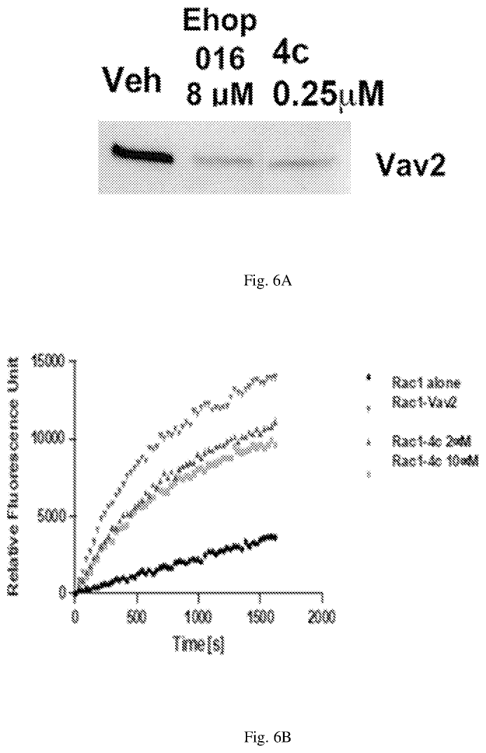

Next, it was identified that GEF was inhibited by Compound 4c, by incubating Sepharose coupled Rac1 (G15A), which bind GEFs tightly to pulldown the GEFs activated in MDA-MB-231 TNBC cell lysates. Rac1 (15A) Sepharose beads were pre-incubated with vehicle or Compound 4c for 1 hour prior to incubation with the cell lysates containing active GEFs. The pulldowns were Western blotted for Tiam-1, Ect2, p-REX, or Vav2. Results show that Vav2 was the only GEF that was displaced by Compound 4c, similar to its parent molecule EHop-016. FIG. 6A demonstrates ˜80% inhibition of Vav2 binding to Rac1by EHop-016 at 8 μM and 4c at 250 nM, indicating that Compound 4c is ˜30× more active than EHop-016. To examine the effect of Compound 4c on GDP/GTP exchange on Rac1 catalyzed by Vav, the changes in fluorescence resonance energy transfer (FRET) between the fluorescent mant group of mant-GDP or mant-GTP and the conserved tryptophan 56 of Rac1 was monitored by fluorescence (290/440 nm) in the presence or absence of purified Vav2 DH/PH domain. Addition of Compound 4c at 2 or 10 μM inhibited the increase in exchange in the presence of Vav2. Therefore, without being bound by any theory, it may be observed that Compound 4c has the same mechanism of action as EHop-016, but at 30× more potency as a specific Vav/Rac inhibitor.

### Compound 4c and Inhibition of the Oncogenic Rac1B Splice Form of Rac1

Compound 4c was characterized as a breast cancer cell inhibitor using HER2 positive and triple negative breast cancer (TNBC) cells. In FIGS. 5A and 5B, it was shown that compound 4c inhibits Rac activation in MDA-MB-231 human TNBC cells. It was shown that that in the MDA-MB-468 TNBC cell line, which also expresses the oncogenic Rac1B, Compound 4c inhibits both Rac1 and Rac1B activation by GTP incorporation (FIG. 7). Therefore, without being bound by any theory, it may be observed that Compound 4c possesses utility as an anticancer drug in cancers with oncogenic Rac.

### Compound 4c in Comparison with MBQ-167 on Viability and Migration of Pancreatic Cancer Cells

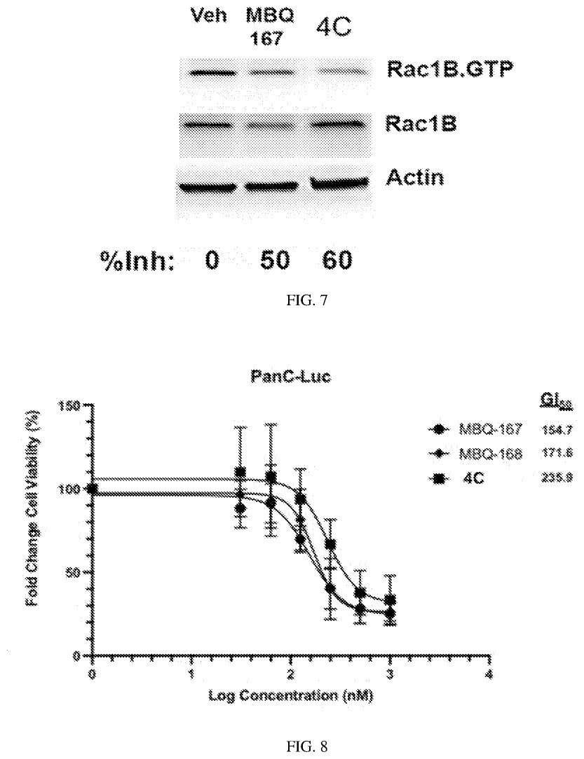

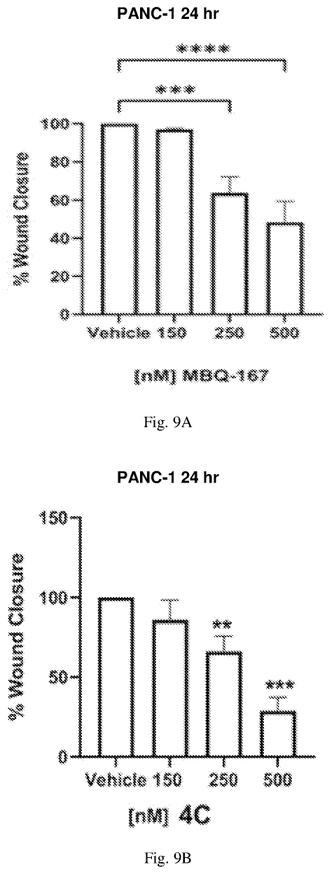

Results from a MTT assay for viability in the invasive pancreatic cancer cell line Panc-1 show that the GIso for Panc-1 cell viability with Compound 4c is 235 nM, while MBQ-167 had a lower GIso of 155 nM (FIG. 8). MBQ-168 has the structure:

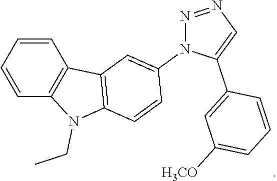 [SMILES: CCn1c2ccccc2c2cc(-n3nncc3-c3cccc(OC)c3)ccc21]

In a wound healing assay for migration at 24 hr, MBQ-167 inhibited Panc-1 cell migration by 40% at 250 nM and 50% at 500 nM. Compound 4c was more effective than MBQ-167 in pancreatic cancer cell migration by a ˜40% at 250 nM and 75% at 500 nM inhibition (FIGS. 9A, 9B). Without being bound by any theory, this data demonstrates that Compound 4c possesses utility as an anti-pancreatic cancer agent.

### Compound 4c on Migration of Macrophage and Fibroblast Cell Lines

Since Rac (Rac2 isoform) is central to macrophage and fibroblast migration during wound healing, and other inflammatory responses during human disease, the effect of Compound 4c on macrophage and fibroblast migration was tested in comparison to MBQ-167.

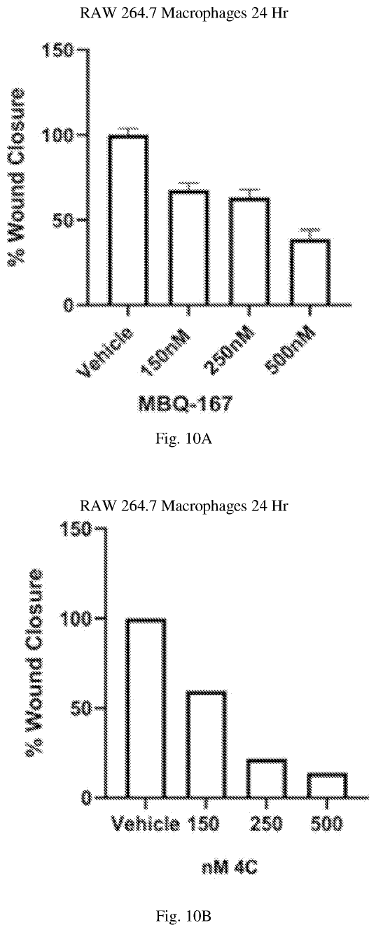

In wound healing assays of a murine macrophage cell line, RAW264.7, MBQ-167 inhibited wound closure by 30% at 150 and 250 nM. Compound 4C inhibited wound closure by 50% at 150 nM, 80% at 250 nM and 90% at 500 nM (FIGS. 10A, 10B).

Therefore, without being bound by any theory, it may be observed that 4c is more effective than MBQ-167 at inhibition of macrophage cell migration by wound healing assays.

Since Rac1 is central to synovial fibroblast migration during Rheumatoid Arthritis progression, the effect of compound 4c was tested on SW-982 synovial cell migration by wound healing assays (FIG. 11).

Taken together, this data may indicate that in addition to its potential utility as an anticancer agent, compound 4c, which is specific to inhibiting the Vav/Rac interaction in monocytes and macrophages may be developed as an anti-inflammatory drug for autoimmune diseases such as lupus, arthritis, multiple sclerosis, systemic sclerosis, type 1 diabetes, inflammatory bowel disease, etc.

### CPV Compound Derivatives 5-9 and Inhibition of Cell Viability of Human Breast Cancer Cells

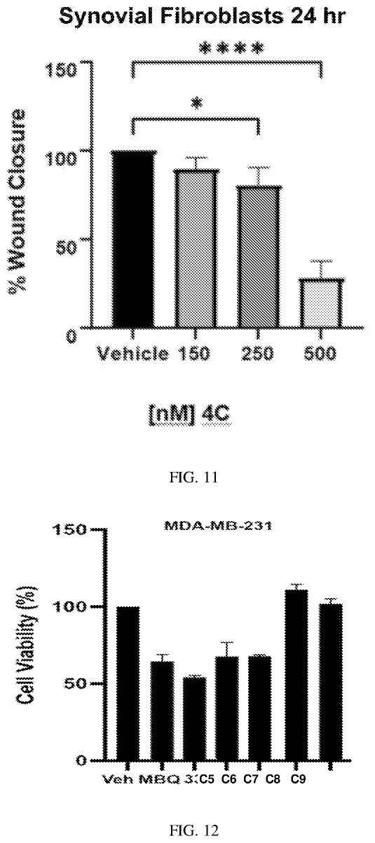

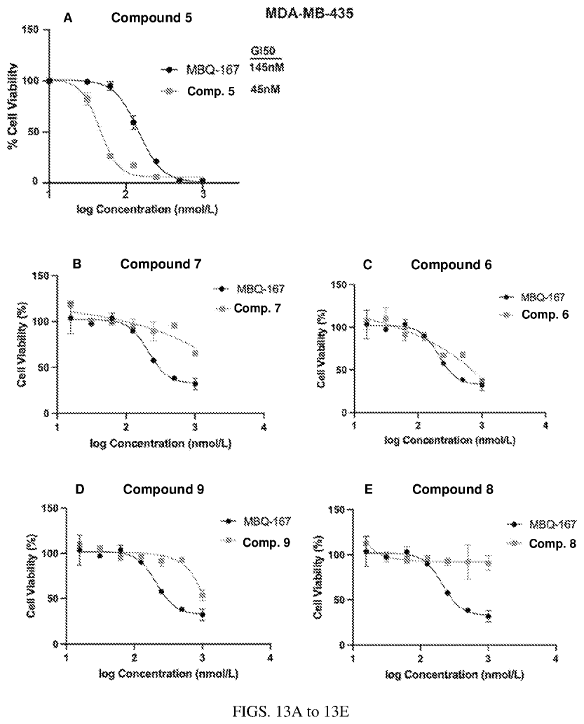

A range of compounds were tested by MTT assays for cell viability in MDA-MB-231 triple negative breast cancer (TNBC) cells and MDA-MB-435 HER2++ cancer cells. The viability of MDA-MB-435 HER2++ highly metastatic cancer cells was quantified following 72 h of MBQ-167 or Compound 5 at concentrations ranging from 0-1000 nM, using a MTT assay (FIG. 12). In this cell line, MBQ-167 treatment resulted in a GI₅₀ of 145 nM for MBQ-167 and a GI₅₀ of 45 nM for Compound 5. Therefore, Compound 5 appears to be ˜3× more cytotoxic than MBQ-167 (FIG. 13A).

Results show that MBQ-167 at 250 nM inhibited cell viability by ˜40%. Compounds 6 and 7 had similar effects, while compound 5 inhibited MDA-MB-435 TNBC cell viability by 50% at 250 nM for 48h (FIGS. 13B, 13C, 13D, 13E). The compounds 6, 7, and 9 reduced the viability of MDA-MB-435, while compound 8 did not have an effect at all concentrations tested. Compound 6 appears to have a similar profile as MBQ-167. Of this class of compounds, Compound 5 appears to be more effective at inhibition of cell viability.

### CPV Compound Derivatives 5-9 and Inhibition of Rac Activation of Human Breast Cancer Cells

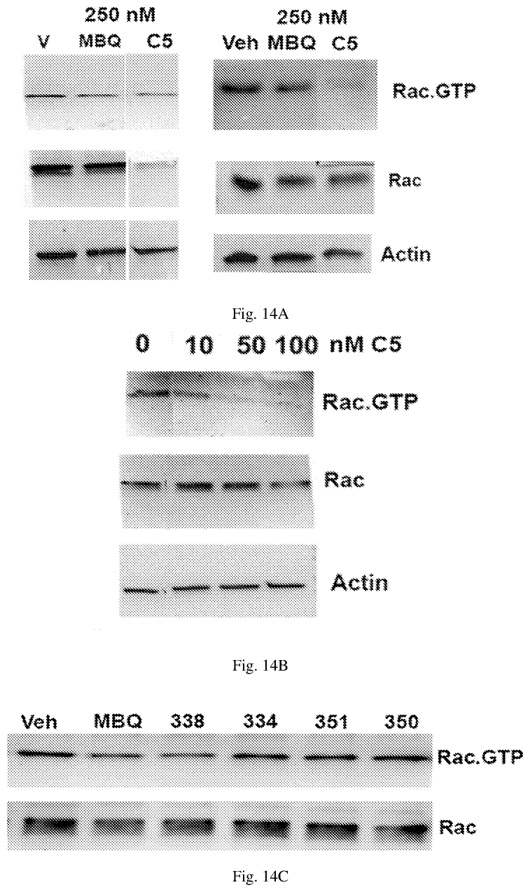

The HER2 positive MDA-MB-435 cell line that has high Rac activity was also used to determine the effect of compound 5 (C5) on Rac activation. MDA-MB-435 cells were incubated with MBQ-167 or CPV compounds at 250 nM for 24 hours and the Rac.GTP was pulled down using a GST-fusion protein of the CRIB domain of P21-activated kinase (PAK), which specifically binds GTP bound active Rac. Results show that similar to MBQ-167, which inhibited Rac activation by ˜60%, compound 5 inhibits Rac activation at a higher percentage (˜90%) than MBQ-167 at 250 nM, without affecting actin (control) expression (FIG. 14A). At concentrations below the 100 nM IC₅₀ of MBQ-167, compound 5 inhibits Rac.GTP loading by ˜30% at 10 nM and >90% at 50 and 100 nM (FIG. 14B).

From the compounds tested, compounds 6 and 7 inhibited Rac activation by ˜60-70% at 250 nM and were not as effective as compound 5 at inhibition of Rac activity (FIG. 14C). Therefore, Compound 5 appears to be a compound with higher inhibitory efficacy than MBQ-167 in metastatic cancer.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A compound of the formula (I), 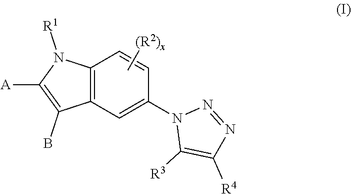
or a pharmaceutically acceptable salt thereof,
wherein A and B taken together with the atom to which they are attached form aryl, cycloalkyl, heterocycloalkyl, or heteroaryl; or A and B are independently H, deuterium, halogen, alkyl, alkenyl, alkynyl, cycloalkyl, heterocycloalkyl, aryl, or heteroaryl, —OH, —CN, amino, amide, alkoxy, carboxy, ester, thioether, sulfoxide, sulfone, sulfonamide;
R¹ is alkyl, H, deuterium, alkenyl, alkynyl, cycloalkyl, heterocycloalkyl, aryl, or heteroaryl;
each R² is independently deuterium, halogen, alkyl, alkenyl, alkynyl, cycloalkyl, aryl, —OH, —CN, amino, amide, alkoxy, carboxy, ester, thioether, sulfoxide, sulfone, or sulfonamide;
R³ is aryl, halogen, alkyl, alkenyl, alkynyl, cycloalkyl, heterocycloalkyl, heteroaryl, aralkyl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, carboxy, ester, thioether, sulfoxide, sulfone, or sulfonamide;
R⁴ is aralkyl, halogen, alkyl, alkenyl, alkynyl, cycloalkyl, heterocycloalkyl, aryl, heteroaryl, hetaralkyl, hydroxyalkyl, carbocyclylalkyl, heterocyclylalkyl, alkoxyalkyl, aminoalkyl, aryl-(alkoxy), aryl-(aryl), —OH, —CN, amino, amide, alkoxy, carboxy, ketone, ester, thioether, sulfoxide, sulfone, or sulfonamide;
x is 0, 1, 2, or 3.

2. The compound of claim 1, wherein A and B taken together with the atom to which they are attached form cycloalkyl, aryl (e.g., C₆-C₁₀ aryl), heterocycloalkyl, or heteroaryl.

3. The compound of claim 1, wherein the compound is of the formula Ia, 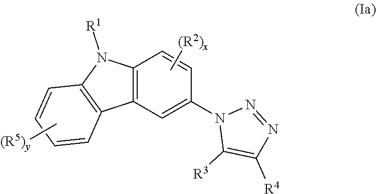
or a pharmaceutically acceptable salt thereof, wherein each R⁵ is independently deuterium, halogen, alkyl, alkenyl, alkynyl, cycloalkyl, aryl, —OH, —CN, nitro, amino, amide, alkoxy, carboxy, ester, thioether, sulfoxide, sulfone, or sulfonamide; and
y is 0, 1, 2, 3, or 4.

4. The compound of claim 1, wherein R³ is aryl (e.g., C₆-C₁₀ aryl such as phenyl).

5. The compound of claim 1, wherein R³ is phenyl, which is optionally substituted by R^{a} (e.g., one R^{a}), wherein each R^{a} is deuterium, halogen, alkyl, alkenyl, alkynyl, cycloalkyl, heterocycloalkyl, aryl, heteroaryl, —OH, —CN, nitro, amino, amide, alkoxy, carboxy, ester, thioether, sulfoxide, sulfone, or sulfonamide.

6. The compound of claim 1, wherein R³ is unsubstituted phenyl.

7. The compound of claim 1, wherein R⁴ is aralkyl (e.g., C₁-C₆ alkyl-aryl), hetaralkyl (e.g., C₁-C₆ alkyl-heteroaryl), hydroxyalkyl, —CN, amide, carboxy, ketone, or ester.

8. The compound of claim 1, wherein R⁴ is C₁-C₆ alkyl-aryl or C₁-C₆ alkyl-heteroaryl, wherein C₁-C₆ alkyl is optionally substituted by deuterium, halogen, alkyl, alkenyl, alkynyl, cycloalkyl, heterocycloalkyl, aryl, or heteroaryl, —OH, —CN, nitro, amino, amide, alkoxy, carboxy, ester, thioether, sulfoxide, sulfone, or sulfonamide.

9. The compound of claim 1, wherein R⁴ is C₁-C₆ alkyl-aryl or C₁-C₆ alkyl-heteroaryl, wherein C₁-C₆ alkyl is optionally substituted by —OH (e.g., hydroxymethyl-aryl or hydroxymethyl-heteroaryl).

10. The compound of claim 1, wherein the compound is of the formula Ib, 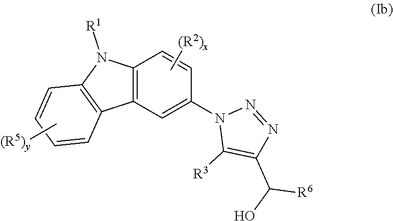
or a pharmaceutically acceptable salt thereof, wherein each R⁵ is independently deuterium, halogen, alkyl, alkenyl, alkynyl, cycloalkyl, aryl, —OH, —CN, nitro, amino, amide, alkoxy, carboxy, ester, thioether, sulfoxide, sulfone, or sulfonamide;
y is 0, 1, 2, 3, or 4; and
R⁶ is aryl or heteroaryl.

11.-20. (canceled)

21. The compound of claim 1, wherein R⁴ is selected from the group consisting of 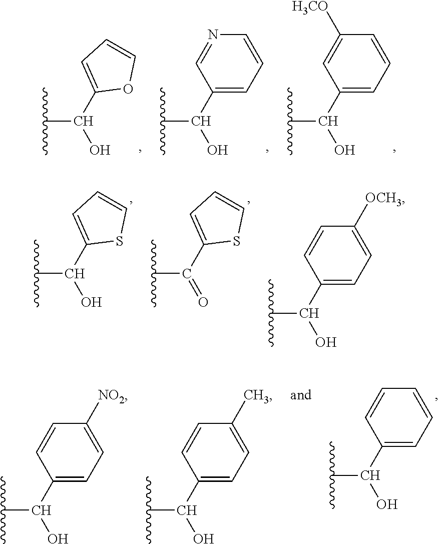
wherein “ [symbol] ” is a point of covalent attachment to triazole ring.

22. The compound of claim 1, wherein R⁴ is —CN.

23. The compound of claim 1, wherein the compound is of the formula (Ic) 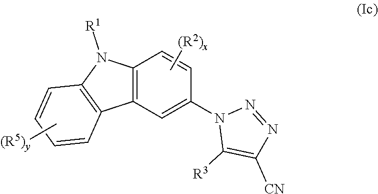
or a pharmaceutically acceptable salt thereof, wherein each R⁵ is independently deuterium, halogen, alkyl, alkenyl, alkynyl, cycloalkyl, aryl, —OH, —CN, nitro, amino, amide, alkoxy, carboxy, ester, thioether, sulfoxide, sulfone, or sulfonamide; and
y is 0, 1, 2, 3, or 4.

24. The compound of claim 1, wherein R⁴ is carboxy (e.g., —C(O) OR⁸, wherein R⁸ is H).

25. The compound of claim 1, wherein the compound is of the formula (Id) 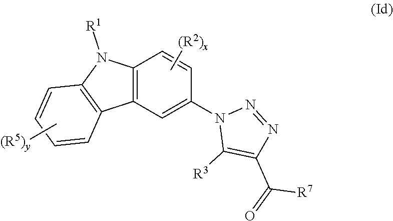
or a pharmaceutically acceptable salt thereof, wherein each R⁵ is independently deuterium, halogen, alkyl, alkenyl, alkynyl, cycloalkyl, aryl, —OH, —CN, nitro, amino, amide, alkoxy, carboxy, ester, thioether, sulfoxide, sulfone, or sulfonamide;
y is 0, 1, 2, 3, or 4,
R⁷ is alkyl, aryl, heteroaryl, —OR⁸ or —NR⁹R¹⁰,
R⁸, R⁹, and R¹⁰ are each independently H, deuterium, alkyl, alkenyl, alkynyl, cycloalkyl, heterocycloalkyl, aryl, heteroaryl, aralkyl, hetaralkyl, hydroxyalkyl, carbocyclylalkyl, heterocyclylalkyl, alkoxyalkyl, aminoalkyl; or R⁹ and R¹⁰ taken together with the atom to which they are attached form heterocycloalkyl or heteroaryl.

26.-37. (canceled)

38. The compound of claim 1, selected from the group consisting of 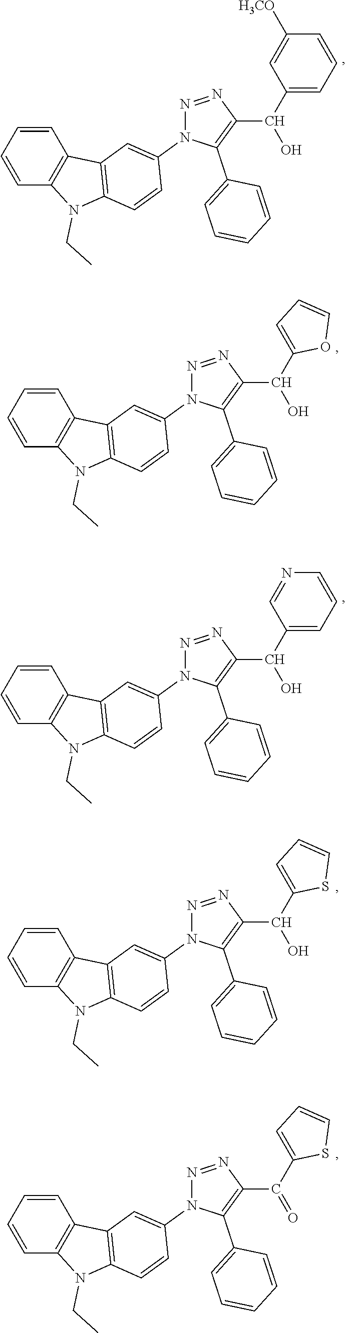 [SMILES: CCn1c2ccccc2c2cc(-n3nnc(C(O)c4cccc(OC)c4)c3-c3ccccc3)ccc21 ; CCn1c2ccccc2c2cc(-n3nnc(C(O)c4ccco4)c3-c3ccccc3)ccc21 ; CCn1c2ccccc2c2cc(-n3nnc(C(O)c4cccnc4)c3-c3ccccc3)ccc21 ; CCn1c2ccccc2c2cc(-n3nnc(C(O)c4cccs4)c3-c3ccccc3)ccc21 ; CCn1c2ccccc2c2cc(-n3nnc(C(=O)c4cccs4)c3-c3ccccc3)ccc21] 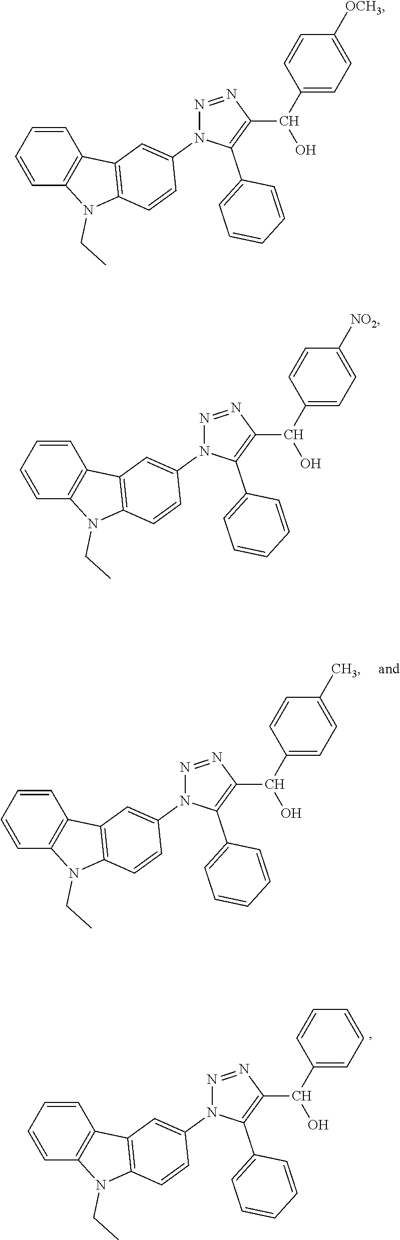 [SMILES: CCn1c2ccccc2c2cc(-n3nnc(C(O)c4ccc(OC)cc4)c3-c3ccccc3)ccc21 ; CCn1c2ccccc2c2cc(-n3nnc(C(O)c4ccc([N+](=O)[O-])cc4)c3-c3ccccc3)ccc21 ; CCn1c2ccccc2c2cc(-n3nnc(C(O)c4ccc(C)cc4)c3-c3ccccc3)ccc21 ; CCn1c2ccccc2c2cc(-n3nnc(C(O)c4ccccc4)c3-c3ccccc3)ccc21]
or a pharmaceutically acceptable salt thereof.

39. The compound of claim 1, selected from the group consisting of 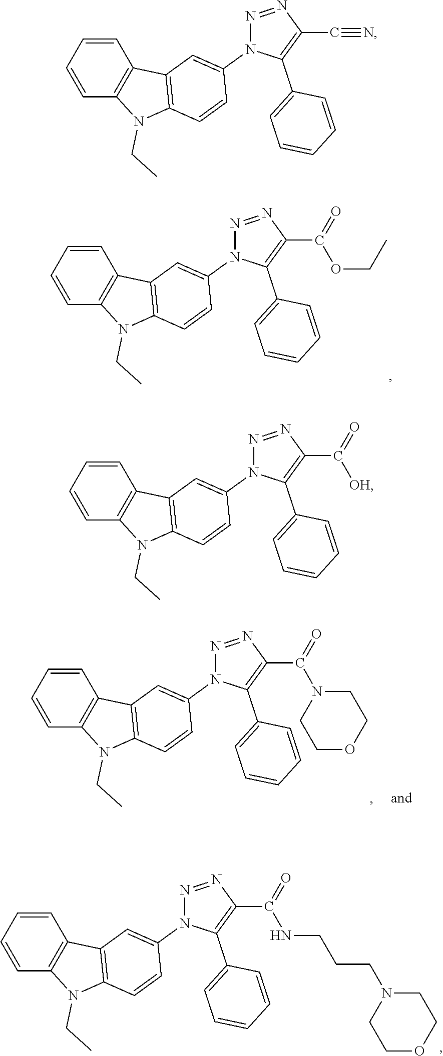 [SMILES: CCn1c2ccccc2c2cc(-n3nnc(C#N)c3-c3ccccc3)ccc21 ; CCOC(=O)c1nnn(-c2ccc3c(c2)c2ccccc2n3CC)c1-c1ccccc1 ; CCn1c2ccccc2c2cc(-n3nnc(C(=O)O)c3-c3ccccc3)ccc21 ; CCn1c2ccccc2c2cc(-n3nnc(C(=O)N4CCOCC4)c3-c3ccccc3)ccc21 ; CCn1c2ccccc2c2cc(-n3nnc(C(=O)NCCCN4CCOCC4)c3-c3ccccc3)ccc21]
or a pharmaceutically acceptable salt thereof.

40. A pharmaceutical composition comprising a compound according to claim 1 and a pharmaceutically acceptable excipient.

41. A method of treating a disease in a patient, the method comprising administering to the patient in need thereof an effective amount of a compound according to claim 1.

42. The method of claim 41, wherein the disease is cancer.

43.-44. (canceled)
